SEQUENCE LISTING (24 sequences)

SEQ ID NO: 1 - DNA, 1894 bp, Homo sapiens
  CDS (183)..(1190): TWIK-1
gggcaggaagacggcgctgcccggaggagcggggcgggcgggcgcgcgggggagcgggcggcgggcgggagccaggcccgggcgggggcgggggcggcggggccagaagaggcggcgggccgcgctccggccggtctgcggcgttggccttggctttggctttggcggcggcggtggagaagatgctgcagtccctggccggcagctcgtgcgtgcgcctggtggagcggcaccgctcggcctggtgcttcggcttcctggtgctgggctacttgctctacctggtcttcggcgcagtggtcttctcctcggtggagctgccctatgaggacctgctgcgccaggagctgcgcaagctgaagcgacgcttcttggaggagcacgagtgcctgtctgagcagcagctggagcagttcctgggccgggtgctggaggccagcaactacggcgtgtcggtgctcagcaacgcctcgggcaactggaactgggacttcacctccgcgctcttcttcgccagcaccgtgctctccaccacaggttatggccacaccgtgcccttgtcagatggaggtaaggccttctgcatcatctactccgtcattggcattcccttcaccctcctgttcctgacggctgtggtccagcgcatcaccgtgcacgtcacccgcaggccggtcctctacttccacatccgctggggcttctccaagcaggtggtggccatcgtccatgccgtgctccttgggtttgtcactgtgtcctgcttcttcttcatcccggccgctgtcttctcagtcctggaggatgactggaacttcctggaatccttttatttttgttttatttccctgagcaccattggcctgggggattatgtgcctggggaaggctacaatcaaaaattcagagagctctataagattgggatcacgtgttacctgctacttggccttattgccatgttggtagttctggaaaccttctgtgaactccatgagctgaaaaaattcagaaaaatgttctatgtgaagaaggacaaggacgaggatcaggtgcacatcatagagcatgaccaactgtccttctcctcgatcacagaccaggcagctggcatgaaagaggaccagaagcaaaatgagccttttgtggccacccagtcatctgcctgcgtggatggccctgcaaaccattgagcgtaggatttgttgcattatgctagagcaccagggtcagggtgcaaggaagaggcttaagtatgttcatttttatcagaatgcaaaagcgaaaattatgtcactttaagaaatagctactgtttgcaatgtcttattaaaaaacaacaaaaaaagacacatggaacaaagaagctgtgaccccagcaggatgtctaatatgtgaggaaatgagatgtccacctaaaattcatatgtgacaaaattatctcgaccttacataggaggagaatacttgaagcagtatgctgctgtggttagaagcagattttatacttttaactggaaactttggggtttgcatttagatcatttagctgatggctaaatagcaaaatttatatttagaagcaaaaaaaaaaagcatagagatgtgttttataaataggtttatgtgtactggtttgcatgtacccacccaaaatgattatttttggagaatctaagtcaaactcactatttataatgcataggtaaccattaactatgtacatataaagtataaatatgtttatattctgtacatatggtttaggtcaccagatcctagtgtagttctgaaactaagactatagatattttgtttcttttgatttctctttatactaaagaatccagagttgctacaataaaataaggggaataataaaaaaaaaaaaaaa

SEQ ID NO: 2 - protein, 336 aa, Homo sapiens
  TWIK-1
MLQSLAGSSCVRLVERHRSAWCFGFLVLGYLLYLVFGAVVFSSVELPYEDLLRQELRKLKRRFLEEHECLSEQQLEQFLGRVLEASNYGVSVLSNASGNWNWDFTSALFFASTVLSTTGYGHTVPLSDGGKAFCIIYSVIGIPFTLLFLTAVVQRITVHVTRRPVLYFHIRWGFSKQVVAIVHAVLLGFVTVSCFFFIPAAVFSVLEDDWNFLESFYFCFISLSTIGLGDYVPGEGYNQKFRELYKIGITCYLLLGLIAMLVVLETFCELHELKKFRKMFYVKKDKDEDQVHIIEHDQLSFSSITDQAAGMKEDQKQNEPFVATQSSACVDGPANH

SEQ ID NO: 3 - DNA, 2514 bp, Homo sapiens
  CDS (126)..(1307): TASK
tgccctgcgcggatagcggcgagcgcagccatgccccaggccgcctccggggcagcagcagcggcggccggggccgatgcgcgggccgggggcgccggggggccggcggcggcccgggcgggacgatgaagcggcagaacgtgcgcacgctggcgctcatcgtgtgcaccttcacctacctgctggtgggcgccgcggtcttcgacgcgctggagtcggagcccgagctgatcgagcggcagcggctggagctgcggcagcaggagctgcgggcgcgctacaacctcagccagggcggctacgaggagctggagcgcgtcgtgctgcgcctcaagccgcacaaggccggcgtgcagtggcgcttcgccggctccttctacttcgccatcaccgtcatcaccaccatcggctacgggcacgcggcacccagcacggatggcggcaaggtgttctgcatgttctacgcgctgctgggcatcccgctcacgctcgtcatgttccagagcctgggcgagcgcatcaacaccttggtgaggtacctgctgcaccgcgccaagaaggggctgggcatgcggcgcgccgacgtgtccatggccaacatggtgctcatcggcttcttctcgtgcatcagcacgctgtgcatcggcgccgccgccttctcccactacgagcactggaccttcttccaggcctactactactgcttcatcaccctcaccaccatcggcttcggcgactacgtggcgctgcagaaggaccaggccctgcagacgcagccgcagtacgtggccttcagcttcgtctacatccttacgggcctcacggtcatcggcgccttcctcaacctcgtggtgctgcgcttcatgaccatgaacgccgaggacgagaagcgcgacgccgagcaccgcgcgctgctcacgcgcaacgggcaggcgggcggcggcggagggggtggcagcgcgcacactacggacaccgcctcatccacggcggcagcgggcggcggcggcttccgcaacgtctacgcggaggtgctgcacttccagtccatgtgctcgtgcctgtggtacaagagccgcgagaagctgcagtactccatccccatgatcatcccgcgggacctctccacgtccgacacgtgcgtggagcagagccactcgtcgccgggagggggcggccgctacagcgacacgccctcgcgacgctgcctgtgcagcggggcgccacgctccgccatcagctcggtgtccacgggtctgcacagcctgtccaccttccgcggcctcatgaagcgcaggagctccgtgtgactgccccgagggacctggagcacctgggggcgcgggcgggggacccctgctgggaggccaggagactgcccctgctgccttctgcccagtgggaccccgcacaacatccctcaccactctcccccagcacccccatctccgactgtgcctgcttgcaccagccggcaggaggccgggctctgaggacccctggggcccccatcggagccctgcaaattccgagaaatgtgaaacttggtggggtcagggaggaaaggcagaagctgggagcctcccttccctttgaaaatctaagaagctcccagtcctcagagaccctgctggtaccacaccccaccttcggaggggacttcatgttccgtgtacgtttgcatctctatttatacctctgtcctgctaggtctcccaccttcccttggttccaaaagccagggtgtctatgtccaagtcacccctactcagccccactccccttcctcatccccagctgtgtctcccaacctcccttcgtgttgttttgcatggctttgcagttatggagaaagtggaaacccagcagtccctaaagctggtccccagaaagcaggacagaaagaaggagggacaggcaggcagcaggaggggcgagctgggaggcaggaggcagcggcctgtcagtctgcagaatggtcgcactggaggttcaagctaactggcctccagccacattctcatagcaggtaggacttcagccttccagacactgcccttagaatctggaacagaagacttcagactcaccataattgctgataattacccactcttaaatttgtcgagtgatttttagcctctgaaaactctatgctggccactgattcctttgagtctcacaaaaccctacttaggtcatcagggcaggagttctcactcccattttacagatgaacctgtattcccaacacttttggaggctgaggttggaggattgcttgagcccaggaattcgagaccagcctaggtgacatagtgagaccccatctctacaaaaaataaaaaattaaccaggtgtggtggcacgtgcctgggagtcccagcgacttgggaggctgaggtgggaggattgtttgagcctgggaggtcgaggctgtagtgagccctgattgcaccactgtactccagcctgggtgacagggcaagaccctgtctcaaaaaaaaaaaaaaa

SEQ ID NO: 4 - protein, 394 aa, Homo sapiens
  TASK
MKRQNVRTLALIVCTFTYLLVGAAVFDALESEPELIERQRLELRQQELRARYNLSQGGYEELERVVLRLKPHKAGVQWRFAGSFYFAITVITTIGYGHAAPSTDGGKVFCMFYALLGIPLTLVMFQSLGERINTLVRYLLHRAKKGLGMRRADVSMANMVLIGFFSCISTLCIGAAAFSHYEHWTFFQAYYYCFITLTTIGFGDYVALQKDQALQTQPQYVAFSFVYILTGLTVIGAFLNLVVLRFMTMNAEDEKRDAEHRALLTRNGQAGGGGGGGSAHTTDTASSTAAAGGGGFRNVYAEVLHFQSMCSCLWYKSREKLQYSIPMIIPRDLSTSDTCVEQSHSSPGGGGRYSDTPSRRCLCSGAPRSAISSVSTGLHSLSTFRGLMKRRSSV

SEQ ID NO: 5 - protein, 405 aa, Murine
  TASK
ENVRTLALIVCTFTYLLVGAAVFDALESEPEMIERQRLELRQLELRARYNLSEGGYEELERVVLRLKPHKAGVQWRFAGSFYFAITVITTIGYGHAAPSTDGGKVFCMFYALLGIPLTLIMFQSLGERINTFVRYLLHRAKRGLGMRHAEVSMANMVLIGFVSCISTLCIGAAAFSYYERWTFFQAYYYCFITLTTIGFGDYVALQKDQALQTQPQYVAFSFVYILTGLTVIGAFLNLVVLRFMTMNAEDEKRDAEHRALLTHNGQAVGLGGLSCLSGSLGDVRPRDPVTCAAAAGGVGVGVGGSGFRNVYAEVLHFQSMCSCLWYKSREKLQYSIPMIIPRDLSTSDTCVEHSHSSPGGGGRYSDTPSHPCLCSGTQRSAISSVSTGLHSLAAFRGLMKRRSSV

SEQ ID NO: 6 - protein, 347 aa, Unknown
  Description of Unknown Organism TWIK-1 homolog
MYTDEGEYSGDTDHGGSTMQKMSPNTRQNFRQNVNVVVCLSAAITLLVFNLIGAGIFYLAETQNSSESLNENSEVSKCLHNLPIGGKITAEMKSKLGKCLTKSSRIDGFGKAIFFSWTLYSTVGYGSLYPHSTLGRYLTIFYSLLMIPVFIAFKFEFGTFLAHFLVVVSNRTRLAVKKAYYKLSQNPENAETPSNSLQHDYLIFLSSLLLCSISLLSSSALFSSIENISYLSSVYFGIITMFLIGIGDIVPTNLVWFSGYCMLFLISDVLSNQIFYFCQARVRYFFHILARKILLLREEDDGFQLETTVSLQHIPIINSQCMPSLVLDCEKEELDNDEKLISSLTST

SEQ ID NO: 7 - protein, 383 aa, Unknown
  Description of Unknown Organism TWIK-1 homolog
MTVSMEENSKIQMLSATSKDKKVATDRSLLNKYHLGPLALHTGLVLSCVTYALGGAYLFLSIEHPEELKRREKAIREFQDLKQQFMGNITSGIENSEQSIEIYTKKLILMLEDAHNAHAFEYFFLNHEIPKDMWTFSSALVFTTTTVIPVGYGYIFPVSAYGRMCLIAYALLGIPLTLVTMADTGKFAAQLVTRWFGDNNMAIPAAIFVCLLFAYPLVVGFILCSTSNITYLDSVYFSLTSIFTIGFGDLTPDMNVIHMVLFLAVGVILVTITLDIVAAEMIDRVHYMGRHVGKAKELAGKMFQLAQSLNMKQGLVSGVGQLHALARFGMLVGREEVDKTQEDGIIAFSPDVMDGLEFMDTLSIYSRRSRRSAENSARNLFLS

SEQ ID NO: 8 - protein, 370 aa, Murine
  TREK-1
MAAPDLLDPKSAAQNSKPRLSFSSKPTVLASRVESDSAINVMKWKTVSTIFLVVVLYLIIGAAVFKALEQPQEISQRTTIVIQKQTFIAQHACVNSTELDELIQQIVAAINAGIIPLGNSSNQVSHWDLGSSFFFAGTVITTIGFGNISPRTEGGKIFCIIYALLGIPLEGFLLAGVGDQLGTIFGKGIAKVEDTFIKWNVSQTKIRIISTIIFILFGCVLFVALPAVIFKHIEGWSALDAIYFVVITLTTIGFGDYVAGGSDIEYLDFYKPVVWFWILVGLAYFAAVLSMIGDWLRVISKKTKEEVGEFRAHAAEWTANVTAEFKETRRRLSVEIYDKFQRATSVKRKLSAELAGNHNQELTPCMRTCL

SEQ ID NO: 9 - protein, 27 aa, Homo sapiens
  TWIK-1 P1
FTSALFFASTVLSTTGYGHTVPLSDGG

SEQ ID NO: 10 - protein, 27 aa, Homo sapiens
  TWIK-1 P2
FLESFYFCFISLSTIGLGDYVPGEGYN

SEQ ID NO: 11 - protein, 27 aa, Unknown
  Description of Unknown Organism P domain of representative K+ channel sequence
YFNCIYFCFLCLLTIGYGDYAPRTGAG

SEQ ID NO: 12 - protein, 27 aa, Unknown
  Description of Unknown Organism P domain of representative K+ channel sequence
YGNALYFCTVSLLTVGLGDILPKSVGA

SEQ ID NO: 13 - protein, 27 aa, Unknown
  Description of Unknown Organism P domain of representative K+ channel sequence
YWTCVYFLIVTMSTVGYGDVYCETVLG

SEQ ID NO: 14 - protein, 27 aa, Unknown
  Description of Unknown Organism P domain of representative K+ channel sequence
IPDAFWWAVVTMTTVGYGDMTPVGFWG

SEQ ID NO: 15 - protein, 27 aa, Unknown
  Description of Unknown Organism P domain of representative K+ channel sequence
IPEAFWWAGITMTTVGYGDICPTTALG

SEQ ID NO: 16 - protein, 27 aa, Unknown
  Description of Unknown Organism P domain of representative K+ channel sequence
IPAAFWYTIVTMTTLGYGDMVPETIAG

SEQ ID NO: 17 - protein, 27 aa, Unknown
  Description of Unknown Organism P domain of representative K+ channel sequence
IPLGLWWALVTMTTVGYGDMAPKTYIG

SEQ ID NO: 18 - protein, 27 aa, Unknown
  Description of Unknown Organism P domain of representative K+ channel sequence
YVTALYWSITTLTTTGYGDFHAENPRE

SEQ ID NO: 19 - protein, 27 aa, Unknown
  Description of Unknown Organism P domain of representative K+ channel sequence
YVTSMYWSITTLTTVGYGDIHPVNTKE

SEQ ID NO: 20 - protein, 27 aa, Unknown
  Description of Unknown Organism P domain of representative K+ channel sequence
YVTALYFTMTCMTSVGFGNVAAETDNE

SEQ ID NO: 21 - protein, 27 aa, Unknown
  Description of Unknown Organism P domain of representative K+ channel sequence
MTSAFLFSLETQVTIGYGFRFVTEQCA

SEQ ID NO: 22 - protein, 27 aa, Unknown
  Description of Unknown Organism P domain of representative K+ channel sequence
FTAAFLFSIETQTTIGYGFRCVTDECP

SEQ ID NO: 23 - protein, 27 aa, Unknown
  Description of Unknown Organism P domain of representative K+ channel sequence
FPSAFLFFIETEATIGYGYRYITDKCP

SEQ ID NO: 24 - DNA, 48 bp, Artificial Sequence
  Description of Artificial Sequence antisense oligonucleotide complementary to the partial mouse cDNA sequence of TASK
caccagcaggtaggtgaaggtgcacacgatgagagccaacgtgcgcac

(19) United States
(12) Patent Application Publication
(10) Pub. No.: US 2002/0094558 A1
(43) Pub. Date: Jul. 18, 2002

(54) Family of mammalian potassium channels, their cloning and their use, especially for the screening of drugs

(75) Inventors: Fabrice Duprat, Vallauris (FR); Florian Lesage, Paris (FR); Michel Fink, La Bocca (FR); Michel Lazdunski, Nice (FR)

Correspondence Address: SCHNADER HARRISON SEGAL & LEWIS, LLP, 1600 MARKET STREET, SUITE 3600, PHILADELPHIA, PA, 19103

(73) Assignee: Centre National De La Recherche Scientifique-CNRS

(21) Appl. No.: 09/939,483

(22) Filed: Aug. 24, 2001

Related U.S. Application Data
(62) Division of application No. 09/144,914, filed on Sep. 1, 1998.
Application No. 09/144,914 is a continuation-in-part of application No. 08/749,816, filed on Nov. 15, 1996.
(60) Provisional application No. 60/095,234, filed on Aug. 4, 1998.

(51) Int. Cl. C12N 9/00; C07H 21/04; C12P 21/02; C12N 5/06; C07K 14/435

(52) U.S. Cl. 435/183; 435/69.1; 435/325; 435/320.1; 530/350; 536/23.2

(57) ABSTRACT

This invention relates to a new member of a recently recognized TWIK potassium ⁺ channel family, herein identified as TASK. For TWIK-related acid-sensitive K ⁺ channel. This is the first cloned mammalian channel that produces K⁺ currents that possesses all the characteristics of background conductances. The inventions also relates to various constructs including the TASK or related human potassium channel family, and their uses.

### RELATED APPLICATIONS

This patent application is a continuation-in-part (CIP) of pending application serial No. 08/749,816 Lesage et al., filed in the U.S. on Nov. 15, 1996, attorney docket No., 989.6351, entitled, “New Family of Mammalian Potassium Channels, Their Cloning And Their Use, Especially For The Screening of Drugs” which is incorporated herein by reference in its entirety, which claims the priority filing date of French patent application serial No. 96/01565, filed Feb. 8, 1996.

This application also claims the priority of provisional patent application serial No. 60/095,234, filed on Aug. 4, 1998, attorney docket No. 989.6705, entitled “Task, a Human Background K⁺channel to Sense External PH Variations near Physiological PH” which is also incorporated herein by reference in its entirety.

### BACKGROUND OF THE INVENTION

1. Field of Invention

The present invention relates to a new family of potassium channels. More specifically, the invention relates to the cloning of new human potassium channels that constitute the members of a new functional and structural group of potassium channels. The abundance of these channels and their presence in a large number of tissues are such as to confer on them a fundamental role in the transport of potassium in a large number of types of cells. The properties of the channels suggest highly interesting applications in the physiology of mammals, especially humans.

2. Description of the Related Art

Potassium channels are ubiquitous membrane proteins that form the largest family of ion channels both in term of functions and structures. By determining and modulating the membrane potential, they play a major role in neuronal integration, muscular excitability as well as hormone secretion (Rudy, 1988; Hille, 1992). More than 40 genes encoding K⁺ channel subunits are now identified in mammals. These subunits fall into two structural classes of pore-forming subunits (Shaker and IRK) (Pongs, 1992; Jan and Jan, 1994; Doupnik et al., 1995; Fakler and Ruppersberg, 1996; Kohler et al., 1996) and four structural classes of auxiliary subunits (Kvβ, Kcaβ, SUR and IsK) (Takumi et al., 1988; Knaus et al., 1994; Pongs, 1995; Inagaki et al., 1996). All Shaker-type subunits have a conserved hydrophobic core containing 6 transmembrane segments (TMS). Associations of Shaker-type subunits with accessory subunits such as KvB, KcaB or IsK give rise to voltage-dependent K⁺ channels (Pongs, 1995; Barhanin et al, 1996; Fink et al, 1996a; Sanguinetti et al., 1996) and Ca²⁺-dependent K⁺ channels (MacCobb et al., 1995; MacManus et al., 1995). Subunits of inward rectifier K⁺ channels (IRK) have only two TMS (Doupnik et al., 1995; Lesage et al., 1995; Fakler and Ruppersberg, 1996). Some IRKs give rise to ATP-sensitive K⁺ channels when they are associated with sulfonylurea receptors (SUR) subunits (Inagaki et al., 1996). Despite a very low overall sequence similarity, Shaker and IRK pore-forming subunits share a conserved domain called the P domain. This peculiar motif is an essential element of the K⁺-selective filter of the aqueous pore and is considered as the signature of K⁺ channel-forming proteins (Heginbotham et al., 1994).

In the above-identified parent patent application, there is described a new family of mammalian K⁺ channel subunits. Despite a low sequence similarity between them (less than 28% of amino acid identity), both cloned members of that family (TWIK-1 and TREK-1) possess the same overall structure with four TMS and two P domains (Fink et al., 1996b; Lesage et al., 1996a; Lesage et al., 1997). The conservation of this structure is not associated with a conservation of the functional properties: TWIK-1 gives rise to weakly inward rectifier K⁺ currents (Lesage et al., 1996a) while TREK-1 produces outward rectifier K⁺ currents (Fink et al., 1996b). However, both channels are open at the resting potential and are able to drive the resting membrane potential near the K⁺ equilibrium potential. This common property suggests that these channels control the resting membrane potential in a large set of cell types.

### BRIEF DESCRIPTION OF THE INVENTION

The present invention describes the cloning, the tissue distribution and the expression of a novel human member of this new structural family. This channel, called TASK for TWIK-related Acid-Sensitive K⁺ channel, as far as is known to the inventors is the first cloned mammalian channel that produces K⁺ currents that possess all the characteristics of background conductances. They are instantaneous with voltage changes and their current-voltage relationships fits the curves predicted from the constant field theory for simple electrodiffusion through an open K⁺-selective pore indicating that TASK currents are voltage-insensitive. The activity of this background channel is strongly dependent on the external pH in the physiological range suggesting that this particular channel is a sensor of external pH variations.

The discovery of this new member of potassium channels and the cloning of the new member of this family provides, notably, new means for screening drugs capable of modulating the activity of these new potassium channels, and thus of preventing or treating the diseases in which these channels are involved.

### BRIEF DESCRIPTION OF NUCLEOTIDE AND AMINO ACID SEQUENCES

The research activities that led to the cloning of the TWIK-1 and TASK channels was carried out in the manner described below with reference to the attached sequences and figures in which:

SEQ ID NO: 1 represents the nucleotide sequence of the cDNA of TWIK1 and its deduced amino acid sequence.

SEQ ID NO: 2 represents the amino acid sequence of the TWIK-1 protein.

SEQ ID NO: 3 represents the nucleotide sequence of the cDNA of TASK and its deduced amino acid sequence.

SEQ ID NO: 4 represents the amino acid sequence of the TASK protein.

### DESCRIPTION OF THE FIGURES

FIG. 1 represents the Northern blot analysis, the nucleotide sequences and the deduced amino acid sequence, as well as the hydrophobicity profile of TWIK-1. (a): expression of TWIK-1 MRNA in human tissues; each track contains 5 μg of poly(A)+RNA; the autoradiograph was exposed for 24 hours. (b) cDNA sequence of TWIK-1 and the amino acid sequences of the coding sequence. The supposed transmembranal segments are circled and the P domains are underlined; □ represents a potential glycosylation site and ▪ represents the threonine residue in the consensus recognition site of protein kinase C. (c): the hydrophobicity analysis and the topology of TWIK-1 deduced from it; the hydrophobicity values were calculated according to the method of Kyte and Doolittle (window size of 11 amino acids) and are presented in relation to the position of the amino acid; the shaded hydrophobic peaks correspond to the transmembranal segments.

FIG. 2 represents the sequence alignments. (a): alignment of the P domains of TWIK-1, TOC/YORK and other representative K⁺ channel families; the identical and conserved residues are circled in black and in gray, respectively. (b): alignment of TWIK-1 with potential homologs of; the sequences M110.2 and F17C8.5 were deduced from the gene sequences (respective access numbers Z49968 and Z35719); the computerized splicing of the other genornic sequences of C. elegans (respective access numbers Z49889, P34411 and Z22180) is not sufficiently precise to allow their perfect alignment and is therefore not shown.

FIG. 3 shows the biophysical and pharmacological properties of K⁺ currents recorded by the imposed voltage technique on Xenopus oocytes that had received an injection of TWIK-1 cRNA; (a): the oocyte was maintained at a holding potential (HP) of −80 mV and the currents were recorded at the end of 1-s voltage jumps from −120 to +60 mV in 20 mV increments. (b): regular current-voltage relationship using the same technique as in (a). (c): potential reversal of the TWIK-1 currents (Eᵣₑᵥ) as a function of the external K+ concentration. (d) current tracings linked to +30 mV depolarizations starting at a holding potential (HP) of −80 mV in the absence (top tracing) and in the presence (bottom tracing) of 1 mM of Ba²⁺. (e): blocking effect of 100 μM of quinine, same protocol as in (d). (f) dose-response relationship of the blocking of the TWIK-1 currents by quinine.

FIG. 4 shows the influence of the expression of TWIK-1 on the membrane potential. (a): dose-response relationships of the cRNA; top row=equilibrium state of the outward currents measured at +30 mV; bottom row membrane potentials associated with the resting state. (b): effect of 100 μM of quinine on the membrane potential of an oocyte which did not receive an injection (left tracing) and that of an oocyte that received 20 ng of TWIK-1 cRNA. (c): statistical evaluation of the depolarizing effects of 100 μM of quinine on oocytes that did not receive injections (left bars) and on oocytes that received injections of 20 ng of TWIK-1 cRNA (right bars); control (unfilled bar), +quinine (solid bars); each bar represents the mean +/− SD of 5 oocytes.

FIG. 5 shows the properties of the single TWIK-1 channel. (a): current tracings recording in the input-output configuration to the membrane potentials indicated in the absence (m) or in the presence (.) of internal M²⁺ (3 mM) and in symmetry with 140 mM of K+. (b): mean of curves I-V (n=10). (c and d): open time of distribution obtained at +80 mV (top histograms) and at −80 mV (bottom histograms) in the presence of 3 mM Mg²⁺ (c) or in the absence of Mg²⁺ (d).

FIG. 6 shows the blocking of the TWIK-1 channels by the internal pH. (a and b): blocking effect of the internal acidification on the TWIK-1 currents, induced by perfusion of CO₂; (a) tracings of superimposed currents induced by a depolarization phase at −30 mV starting at HP=−80 mV, control (top tracing), effect when equilibrium is reached in the presence of CO₂ (bottom tracing); (b): graph (n=5) showing the almost complete blockade of the TWIK-1 currents induced by CO₂; (c and d): internal acidification induced by the application of DNP (1 mM). (c): same protocol as in (a), control (top tracing) and after 5 minutes of application of DNP (bottom tracing); (d): graph (n=4) indicating the percentage of TWIK-1 current remaining after treatment with DNP. (e and f): imposed voltage (method: attached patch) under symmetrical conditions of K+ concentration (140 mM) maintained at +80 mV. (e) course over time of the effect of 1 mM of DNP (marked with arrow) on the activities of the single TWIK-1 channel. (f): graph (n=4) showing the effect of DNP on the mean probability of opening NPₒ calculated during 1 minute of recording starting at the equilibrium state. (g): activities measured in the “inside-out patch” state at 80 mV at different internal pH values. Bar graph (n=10) of NPₒ in relation to the internal pH.

FIG. 7 shows the activation of the TWIK-1 channels by PMA, activator of protein kinase C. (a): perfusion of PMA (30 nM) for 10 minutes increases the TWIK-1 current (top tracing) induced by a depolarization phase at +30 mV starting at HP=−80 mV, control current (top tracing). (b): graph (n=5) showing the activation effect of PMA on the TWIK-1 currents. (c and d): attached patch configuration under symmetrical K+ concentration conditions maintained at +60 mV; (c): course over time of the effect of 30 nM of PMA on the single channel activities; the recordings of the channel activity were performed with a rapid scanning before and after the application of PMA; (d): bar graph (n=5) showing the activation effect of PMA on NPₒ.

FIG. 8 (which consists of 8A and 8B, an enlargement of 8A) show the nucleotide and deduced amino acid sequences of human TASK and partial amino acid sequence of mouse TASK. Consensus sites for N-linked glycosylation (*) and phosphorylation by protein kinase C (n), protein kinase A (s) and tyrosine kinase (l) in human TASK. These sites have been identified by using the prosite server (European Bioinformatics Institute) with the ppsearch software (EMBL Data library) based on the MacPattern program. The sequence of human and mouse TASK have been deposited in the GenBank/EMBL database under the accession numbers AF006823 and AF006824, respectively.

FIG. 9 shows the sequence comparison and membrane topology of TWIK-related channels. A: Alignment of human TWIK-1, mouse TREK-1 and human TASK sequences. Identical and conserved residues are shown in black and grey, respectively. Dashes indicate gaps introduced for a better alignment. Relative positions of putative transmembrane segments (M1 to M4) and P domains (P1 and P2) of human TASK are also indicated. The M1-M4 domains were deduced from a hydropathy profile computed with a window size of 11 amino acids according to the Kyte and Doolittle method (Kyte and Doolittle, 1982). B: Putative membrane topology of TWIK-1, TREK-1 and TASK channels.

FIG. 10 shows the northern blot analysis of TASK distribution in adult human tissues. Human multiple tissues Northern blots from Clontech were probed at high stringency with a TASK cDNA probe. Each lane contains 2 μg of poly(A)⁺RNA. Autoradiograms were exposed for 48 h at −70° C. The blots were re-probed with a β-actin cDNA probe for control. sk. muscle: skeletal muscle, sm. intestine: small intestine, PBL: peripheral blood leukocytes.

FIG. 11 shows the distribution of TASK mRNA in adult mouse. A: Northern blot analysis. Each lane contains 2 μg of poly(A)⁺RNA. Autoradiograms were exposed for 72 h at −70° C. The blots were re-probed with a β-actin cDNA probe for control. B, C, D: In situ hybridization analysis from a coronal section at the level of the forebrain (B), the cerebellum (C), and the heart (D). Warmer colors represent higher levels of expression. CA1-CA3: fields CA1-3 of Ammon's horn, Cx: cerebral cortex, DG: dentate gyrus, Gl: granular layer, Hb: habenula, SN: substantia nigra, PLCo: postero lateral cortical amygdaloid nuclei, PVP: paraventricular thalamic nucleus, A: atrium, V: ventricule.

FIG. 12 shows the biophysical properties of TASK in Xenopus oocytes and COS cells. A: TASK currents recorded from a Xenopus oocyte injected with TASK cRNA and elicited by voltage pulses from −150 mV to +50 mV in 40 mV steps, 500 ms in duration, from a holding potential of −80 mV in low (2 mM K⁺) or high K⁺ solutions (98 mM K⁺). The zero current level is indicated by an arrow. B: Current-voltage relationships. Mean currents were measured over the last 50 ms at the end of voltage pulses from −150 to +50 mV in 10 mV steps as in A. Modified ND96 solutions containing 2 mM K⁺ and 96 mM TMA were used, TMA was then substituted by K⁺ to obtain solutions ranging from 2 mM to 98 mM K⁺. TASK currents are not sensitive to external TMA, no changes were observed upon substitution of NaCl by TMA (data not shown). C: Upper panel: reversal potentials of TASK currents as a function of external K⁺ concentration (mean ±SEM, n=3). Lower panel: slope conductance measured between +10 and +50 mV on current-voltage relations as in A, plotted as a function of the external K⁺ concentration (mean ±SEM, n=3). The mean values were fitted with an hyperbola function. D: Theoretical current-voltage relation in the same conditions as in A calculated according to the following modified Goldman-Hodgkin-Katz (GHK) current relationship:

$${I}_{K +} = {P}_{K +} ( \frac{{[ {K}^{+} ]}_{out}}{{K}_{0.5} + {[ {K}^{+} ]}_{out}} ) ( \frac{{V}_{m} {F}^{2}}{RT} ) \frac{{[ {K}^{+} ]}_{in} - {[ {K}^{+} ]}_{out} {ⅇ}^{- VF / RT}}{1 - {ⅇ}^{- VF / RT}}$$

where I_{K+} is the potassium current, P_{K+} is the apparent permeability for K⁺, K_{0.5} the half maximum activation by K⁺, [K⁺]ₒᵤₜ and [K⁺]ᵢₙ are the external and internal K⁺ concentrations, Vₘ the membrane potential, F, R and T have their usual meanings. The classical GHK relation has been modified with:

$$\frac{{[ {K}^{+} ]}_{out}}{{K}_{0.5} + {[ {K}^{+} ]}_{out}}$$

to take into account the sensitivity of the conductance to external K⁺. E: TASK currents recorded from a transfected COS cell and elicited by voltage pulses from −150 mV to +50 mV in 40 mV steps, 500 ms in duration, from a holding potential of −80 mV, in low (5 mM K⁺) or high K⁺ solutions (155 mM K⁺). The zero current level is indicated by an arrow. F: Current-voltage relationships. Mean currents were measured over the last 50 ms at the end of voltage pulses ranging from −150 to +50 mV in 10 mV steps as in E. Solutions containing 5 mM K⁺ and 150 mM TMA were used, TMA was then substituted by K⁺ to obtain solutions ranging from 5 mM to 155 M K⁺.

FIG. 13 shows the pH dependent regulation of TASK in Xenopus oocytes and COS cells. A: Current-voltage relationships recorded from a TASK-expressing oocyte with a ramp ranging from −150 mV to +50 mV, 500 ms in duration, from a holding potential of −80 mV, in ND96 solution at pH 6.5, 7.4 or 8.4. Inset: Currents elicited by voltage pulses to +50 mV, 500 ms in duration, in the same conditions as above. The zero current level is indicated by an arrow. B: pH-dependence of TASK activity in Xenopus oocyte recorded at −50, 0 and +50 mV (mean ±SEM, n=3) as in A. Data were fitted with a Boltzman relation. C: Current-voltage relation recorded from a TASK-expressing COS cell with a ramp ranging from −150 mV to +50 mV, 500 ms in duration, from a holding potential of −80 mV, in 5 mM K⁺ solution at pH 6.1, 7.4 and 8.4. Inset: Currents elicited by voltage pulses to +50 mV, 500 ms in duration, in the same conditions as above. The zero current level is indicated by an arrow. D: pH-dependence of TASK activity recorded in COS cell at −50, 0 and +50 mV (mean ±SEM, n=3) as in C. Data were fitted with a Boltzman relationship.

### CLONING AND PRIMARY STRUCTURE OF TWIK-1

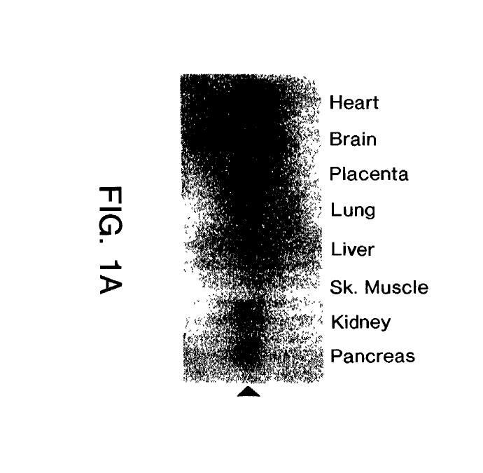

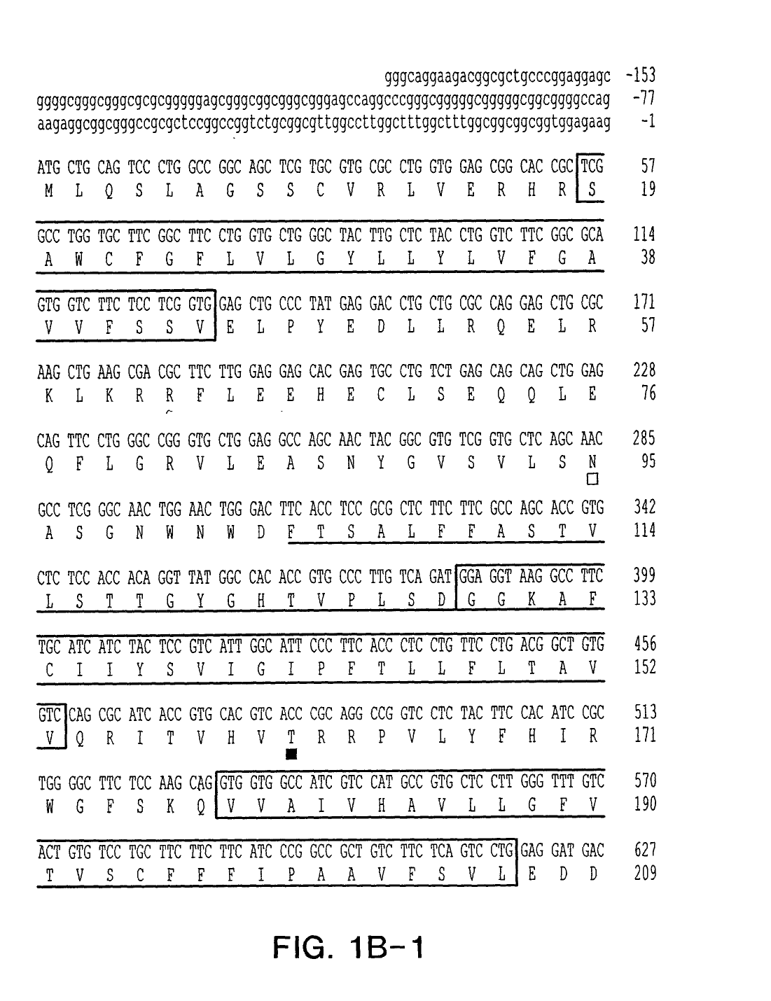

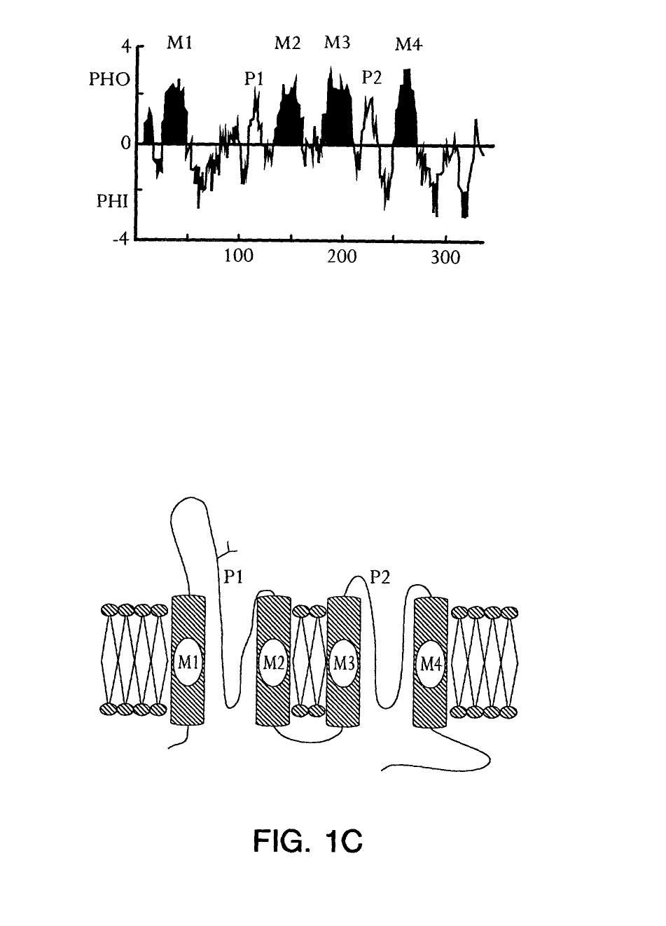

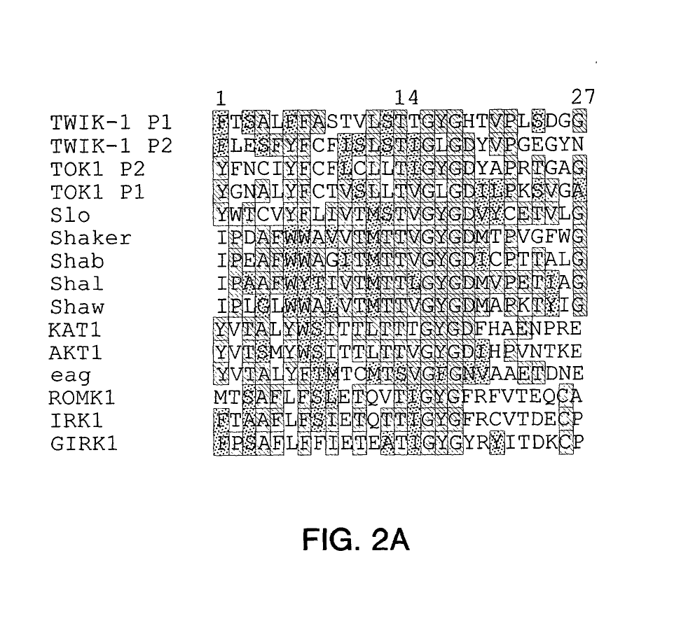

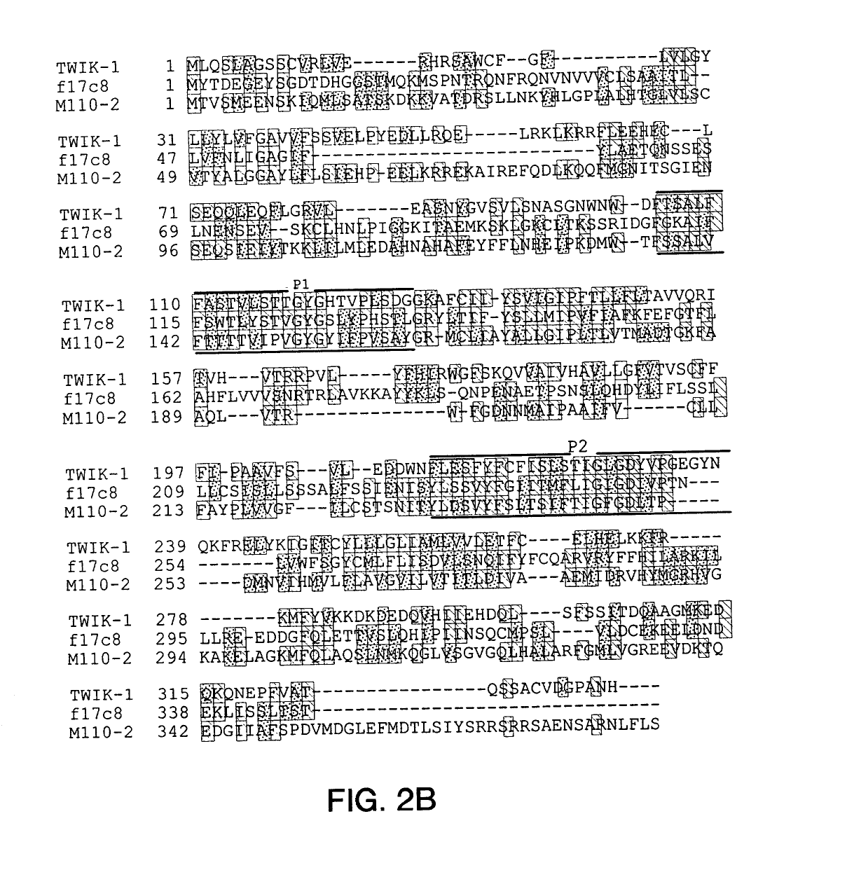

The P domains of K⁺ channels were used to determine the corresponding sequences in the GenBank data base by means of the BLAST sequence alignment program (Altschul, S. F. et al., 1990, J. Mol. Biol., 215, 403-410). There was thus identified a 298 bp human Tag expressed sequence (EST, HSC3AH031), the deduced amino acid sequence of which includes a nonconventional “P-like” domain sequence: GLG in place of GYG as shown in FIG. 2a. It was then envisaged that this EST sequence was a partial copy of a MRNA coding a new type of K+ channel subunit. A DNA probe was prepared from this sequence in order to carry out hybridization with a Northern blot (Clontech) of multiple human tissues. A 1.9 kb transcript was thereby found in abundance, as shown in FIG. 1a, in the heart and the brain and, at lower levels, in the placenta, the lung, the liver and the kidney. The DNA probe was used to screen a bank of kidney CDNA and four independent clones were obtained. The cDNA inserts of 1.8 to 1.9 kb of these clones all have the same open reading frame (ORE) containing a region identical to the 298 bp sequence of HSC3AH031 and differing solely in the length of their noncoding 5′ sequences.

The TWIK-1 coding sequence was amplified using a low-error rate DNA polymerase (Pwo DNA pal, Boehringer) and subcloned in the plasmid pEXO so as to yield pEXO-TWIK-1. Mutations were performed using the whole plasmid pEXO-TWIK-1 with a highly reliable PCR extension kit (Boehringer) and two adjacent primers. One of these introduced a punctiform mutation in the IINIK-1 coding sequence, changing the 161 Thr codon into a codon for alanine. The product of the PCR was linearized by the enzyme BamHI and the cRNA were synthesized using a T7 RNA polymerase (Stratagene). Preparation of the X. larvis oocytes and cRNA injection were carried out in accordance with the literature (Guillemare, E. et al., 1992, Biochemistry, 31, 1246312468.

Primary Structure of TWIK-1

The following characteristics of this K⁺ channel were demonstrated:

The sequences of the cDNA clones contain an ORE of 1011 nucleotides coding for a polypeptide of 336 amino acids shown in FIG. 1b.

The protein has two P domains.

Other than the P domains, no significant alignment was seen between TWIK-1 and a K+ channel recently cloned in yeast and which also has two P domains (Ketchum, K. A. et al., 1995, Nature, 376, 690-695).

Analysis of the hydrophobicity of TWIK-1, shown in FIG. 1c, reveals the presence of four transmembranal domains, designated T1 to T4

By placing the NH₂ end on the cytoplasmic surface, in accordance with the absence of signal peptide, one obtains the topology model shown in FIG. 1c.

In this model, the two P domains are inserted in the membrane from the exterior in accordance with the known orientation of these loops in the K+ channels.

In addition, the general structural unit of TWIK- 1 is similar to the unit that one would obtain by making a tandem of two classical subunits rectifying the entry of a potassium channel. Like a classical inward rectifier, TWIK-1 does not exhibit the highly conserved segment S4 which is responsible for the sensitivity to the membrane potential of the inward rectification of the K+ channels of the Kv family.

An unusual large loop of 59 amino acids is present between M1 and P1, such as to extend the length of the linker M1-P1 of the extracellular side of the membrane.

A potential site of N-glycosylation is present in this loop.

Three consensus sites of phosphorylation are present at the N-terminal (Ser 19 for calcium calmodulin kinase II) and C-terminal (Ser 303 for casein kinase II) ends of the cytoplasmic domains, and in the M2-M3 linker (Thrl61 for protein kinase II).

The alignment of the P domains of an important group of K⁺ channels is presented in FIG. 2a. It shows that the regions constituting the pore selective for K⁺ are well conserved including the G residues in position 16 and 18 and three other residues indicating practically exclusively conservative changes in positions 7, 14 and 17. It is of interest to note that a leucine residue is present in the place of a tyrosine conserved in position 18 in the P2 domain of TWIK-1, or of a phenylalanine in position 17 of the P domain of the K⁺ channel of type eag.

Functional expression of TWIK-1

For the functional study, the coding sequence of TWIK-1 was inserted between the noncoding sequences 5′ and 3′ of Xenopus globin in the vector pEXO (Lingueglia, E. et al., 1993, J. Biol. Chem., 269, 13736-13739). A complementary RNA (cRNA) was transcribed of this construction and injected in the oocytes of X laevis. In a 0.3 ml perfusion chamber, a single oocyte was impaled on two standard glass microelectrodes (0.5 -2.0 MW) charged with 3 M KCl and maintained under voltage-clamp with a Dagan TEV200 amplifier. The bath solution contained 98 mM KCl, 1.8 mM CaCl₂, 2 mM MgCl₂ and 5 mM HEPES at pH 7.4 with KOH. Stimulation of the preparation, data acquisition and analyses were carried out with the pClamp program (Axon Instruments) USA3.

For the patch-clamp experiments, the vitelline membrane was removed from the oocytes as described in the literature (Duprat, F. et al., 1995, Biochem. Biophys. Res. Commun., 212, 657-663); the oocytes were then placed in a bath solution containing 140 M KCl, 1.8 mM CaCl₂, 2 mM MgCl₂ and 5 mM HEPES at pH 7.4 with KOH. The pipettes were filled with a strong K⁺ solution (40 mM KCl, 100 mM of potassium methane sulfonate, 1.8 mM CaCl₂, 2 m M MgCl ₂ and 5 mM HEPES adjusted to pH 7.4 with KOH). 100 μM of GdCl₃ was added to the pipette solution to inhibit the action of the activated channel. The inside-out patches were perfused with a solution containing 140 mM KCl, 10 mM CaCl₂, 5 mM HEPES adjusted to pH 7.2 with KOH and 5 m M EGTA added daily. The single channel signals were filtered at 3.5 kHz and analyzed with the Biopatch program (Big-Logic, Grenoble, France).

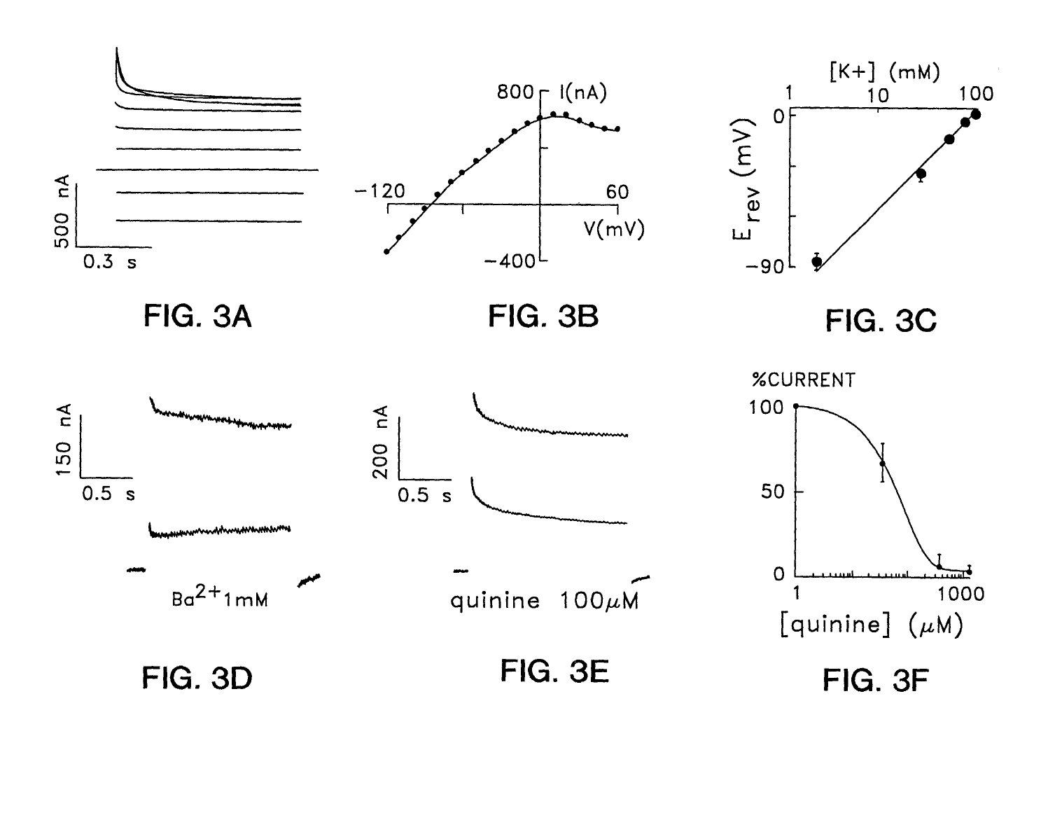

A noninactivating current, free from noninjected cells, was measured by the imposed voltage technique, as shown in FIG. 3a. Kinetic activation of the current is usually instantaneous and cannot be resolved because it is masked by the capacitive discharge of the current recorded at the beginning of the impulse. The current-voltage relationship is linear above 0 mV and then saturates for a stronger depolarization of the membrane, as shown in FIG. 3b. TWIK-1 is therefore K⁺ selective. In the case of a replacement of the external K⁺ by Na+ or N-methyl-D-gluconate, the reversal of the potential of the currents follows the K⁺ equilibrium potential (EK), as shown in FIG. 3c. In addition, a change by 10 in the concentration [(K)]ₒ, leads to a change of 56 +/−2 mV in the inversion value of the potential, in accordance with Nernst's equation.

As shown in FIG. 3, the K⁺ currents of TWIK-1 are inhibited by Ba²⁺ (FIG. 3d) with an IC₅₀ value of 100 μM, by quinine (FIG. 3e and 3f) and by quinidine (not shown) with respective IC ₅₀ values of 50 and 95 μM. The TWIK-1 currents are slightly sensitive to TEA and to the class III antiarrhythmic agent tedisamil (30% inhibition for each, at 20 mM and 100 μM, respectively). Less than 10% inhibition was seen after application of 4-aminopyridine (1 mM), apamin (0.3 μM), charybdotoxine (3 μM), dedrotoxine (0.1 μM), clofilium (30 μM), amiodarone (100 μM) and glibenclamide (30 μM). The TWIK-I channel is not sensitive to the Kit channel openers cromakaline (100 μM) and pinacidil (100 μM).

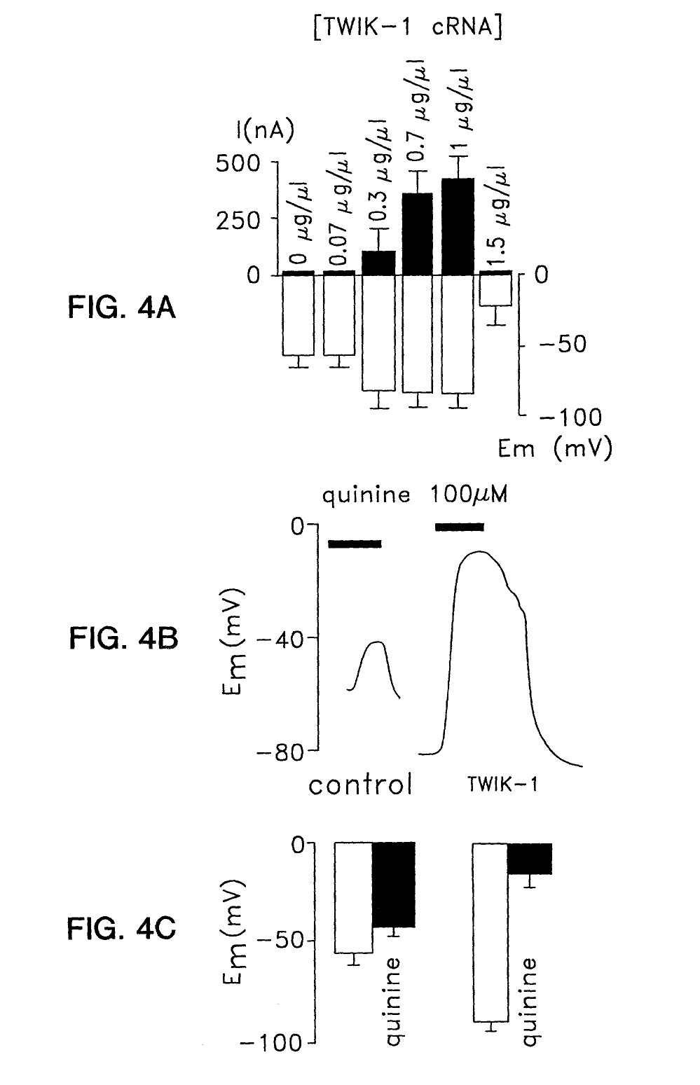

FIG. 4 shows the effect of increasing the doses of injected TWIK-1 cRNA on the independent expression of the time of the K⁺ currents and on the resting state of the membrane potential (Eₘ). As soon as the current appears, the oocytes become increasingly polarized, reaching a value of Eₘ close to E_{K} The amplitude of the TWIK-1 current reaches values of 0.6 to 0.8 μM with the injection of 20 ng per oocyte. Higher doses of TWIK-1 cRNA are toxic, leading to a reduction in expression. In oocytes that received 20 ng of cRNA, quinine is the best blocker of TWIK-1, inducing a noteworthy reversible depolarization (73+/−6 mV, n=5) as shown in FIGS. 4b and 4c.

The unitary properties of the TWIK-1 channel

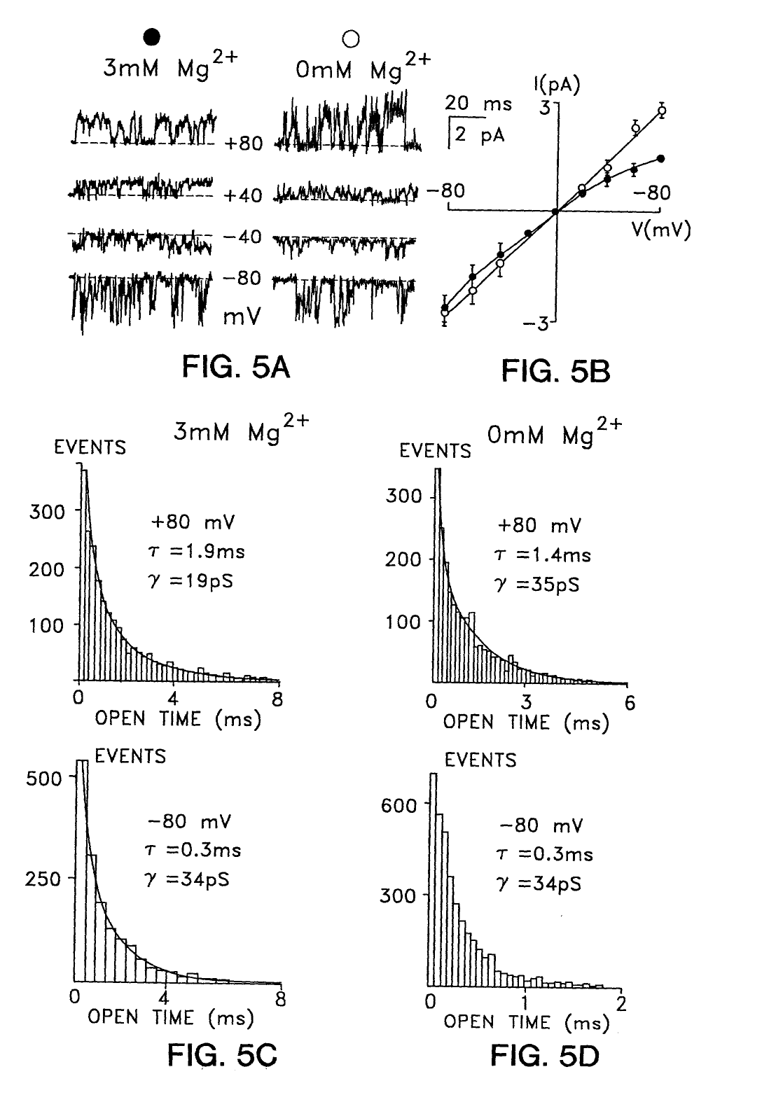

Single channel current recordings, shown in FIG. 5, in an inside-out patch configuration or in a whole cell configuration show that the TWIK-1 channels assure the passage of influx or exit currents as a function, respectively, of a depolarization or a hyperpolarization (FIG. 5a). The current voltage relationship of the single channel, shown in FIG. 5b, shows a barely accentuated inward rectification in the presence of 3 mM (FIG. 5) and 10 mM (not shown) of Mg²⁺ on the cytoplasmic side. As shown in FIG. 5b, this rectification disappears in the absence of internal M²⁺. With 3 mM of internal Mg²⁺, the mean duration of opening at +80 mV is 1.9 ms and the unitary conductance is 19+/−1 pS (FIG. 5c). At −80 mV, the charnels are oscillating with a mean duration of opening of 0.3 ms, and a conductance value in creasing to 34 pS. The withdrawal of the internal Mg²⁺ ions does not influence the kinetic parameters under either polarized or depolarized conditions, but the unitary conductance measured at −80 mV reaches 35+/−4 pS. This apparent increase in conductance in the single channel suggests that it is the extremely rapid oscillation induced by Mg ²⁺ that results in an underestimation of the real value of conductance. The same properties were observed in the fixed cell configuration, showing that the channel behavior is not modified by the excision of the patch. The TWIK- 1 channels in the excised patches do not discharge and do not appear to be deficient in intracellular constituents. In contrast to numerous channels which require the presence of ATP for their activity in the excised patch configuration, ATP is not required for the expression of TWIK-1. In addition, perfusion of the patch with a solution containing 10 mM of ATP does not induce any effect on the activity of the TWIK-1 channel.

Regulation of the TWIK-1 channel.

The intracellular pH (pHᵢ) is involved in the control of numerous cellular processes, and in cells such as the hepatic cells, the change in pHᵢ regulates the membrane potential (Bear, C. E. et al., 1988, Biochim. Biophys. Acta, 944, 113-120).

Intracellular acidification of the oocytes was produced using two methods:

superfusion with a solution enriched in CO₂ which produces acidification by a mechanism involving the bicarbonate transport system (Guillremare, E. et al., 1995, Mol. Pharmacol., 47, 588-594);

treatment with dinitrophenol (DNP), which is a metabolic inhibitor that decouples the H+ gradient in mitochondria and induces internal acidity (Pedersen, P. L. and Carafoli, E., 1987, Trends Biol. Sci., 12, 146-189).

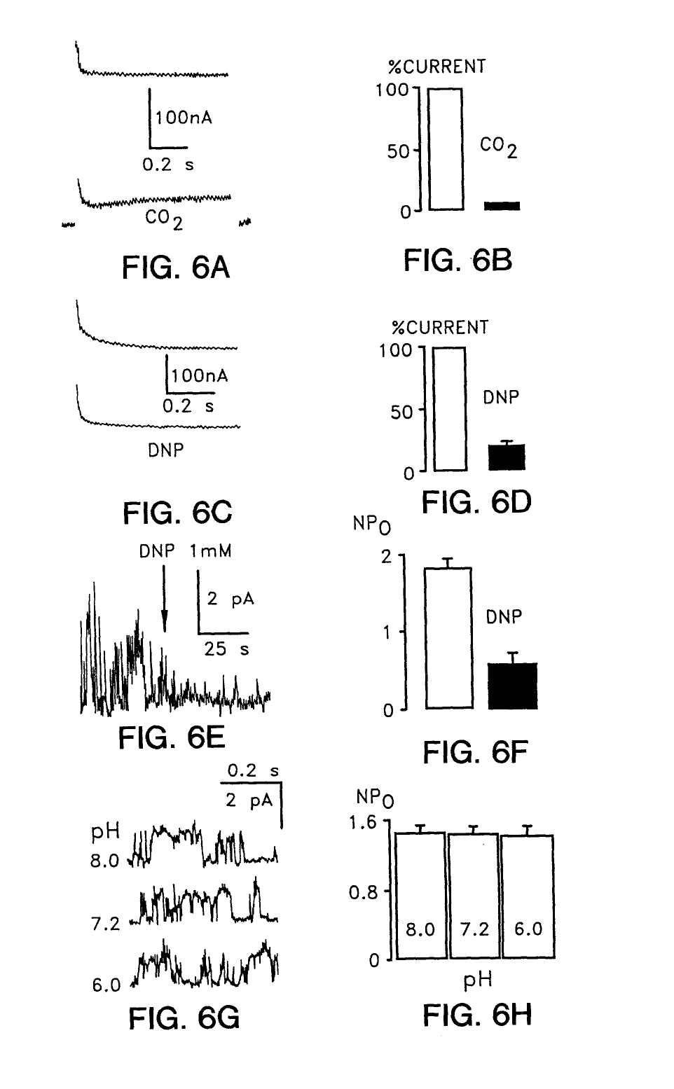

Both of these experimental methods resulted in a significant reduction in the TWIK-1 currents, greater than 95% in the case of CO₂ and 80% in the case of DNP of the control amplitude values, as shown in FIGS. 6a to 6d. The inhibition induced by DNP on the activity of the single K⁺ channel was again observed under the attached patch conditions, as shown in FIGS. 6eto 6f. However, after excision of the patch, the activity of the channel became insensitive to the acidification of the internal solution produced either by modifying the Na₂HPO₄/NaH₂PO₄ buffer ratio (FIGS. 6g and 6h3 or by bubbling of CO₂ (not shown). Thus, the effect of the pH value on the activity of the TWIK-1 channel is probably indirect.

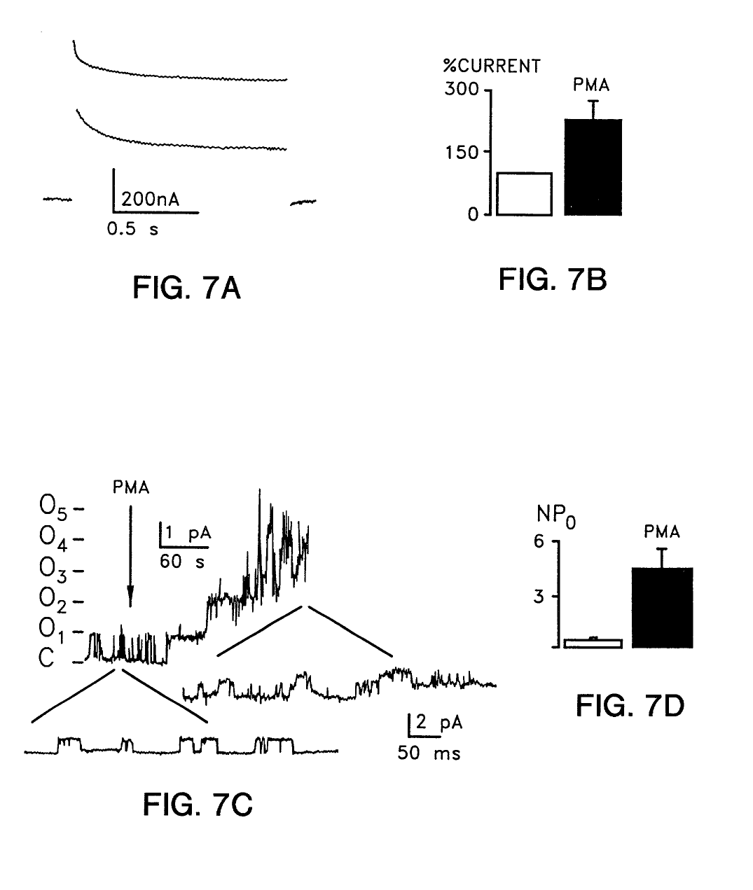

Phosphorylation or dephosphorylation of specific amino acid residues is an important mechanism of regulation of the ionic channels (Levitan, I. B., 1994, Annul Rev. Physiol., 56, 193-212). As shown in FIG. 7, activation of protein kinase C by phorbol-12 myristate acetate (PMA, 30 nM) increases the TWIK-1 currents. The inactive phorbol ester 4α-phorbol-12, 13 didecanoate (PDA, 1 μM) has no effect. In an attached patch which initially expressed solely a single channel, application of PMA showed the presence of at least five channels (FIG. 7c and 7d). This experiment shows that at least four channels are silently present in the patch before the application of PMA. Since the TWIK-1 sequence contains a consensus phosphorylation site for protein kinase C (PKC), located at the level of the threonine in position 161 (FIG. 1b), the effect of PMA suggests regulation under the control of PKC. However, the mutation of the threonine 161 into alanine leads to a muted channel which remains functional and conserves the capacity to be activated by PMA.

Activation of protein kinase A by application of 8-Cl-AMPc (300 μM) or forskolin (10 μM) does not affect the activity of TWIK-1. Elevation of the cytoplasmic Ca²⁺ concentration by application of A23187 (1 μM) which could be activated by Ca2+-calmodulin kinase II and/or reveal the presence of a channel activated by the Ca²⁺, is also without effect on the properties of the TWIK-1 channel.

Cloning and Primary Structure of TASK.

Another Member of the TWIK-related K⁺ Channel Family

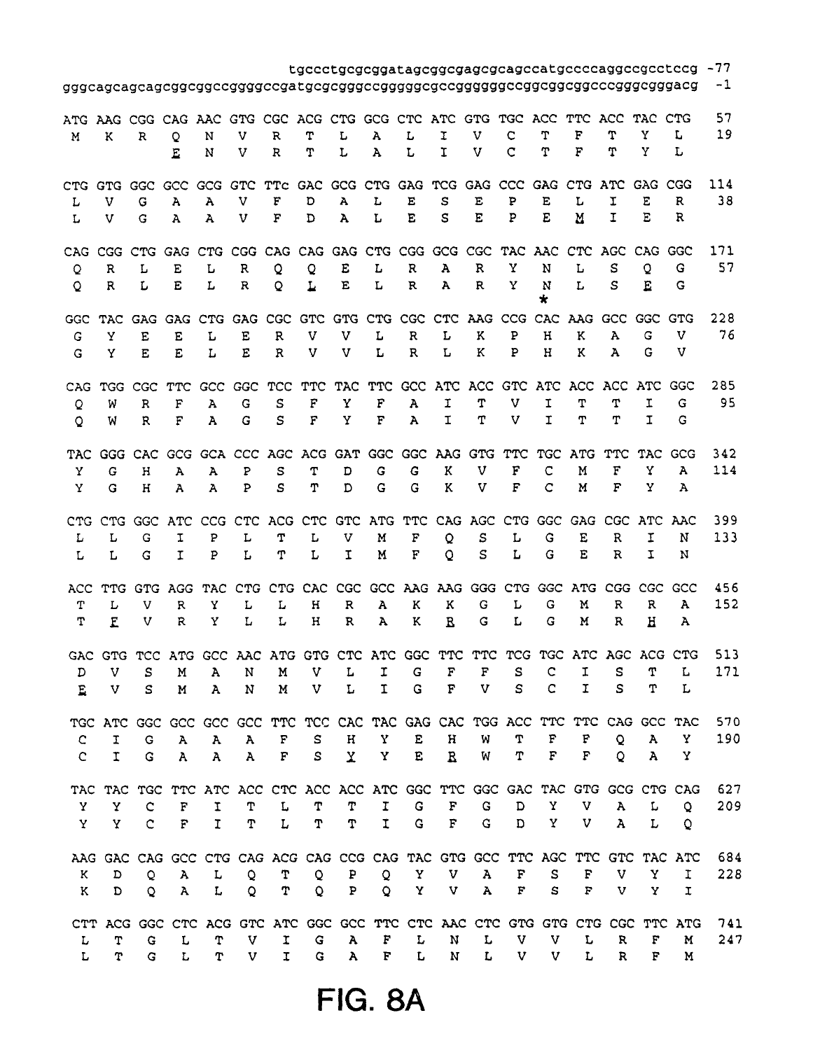

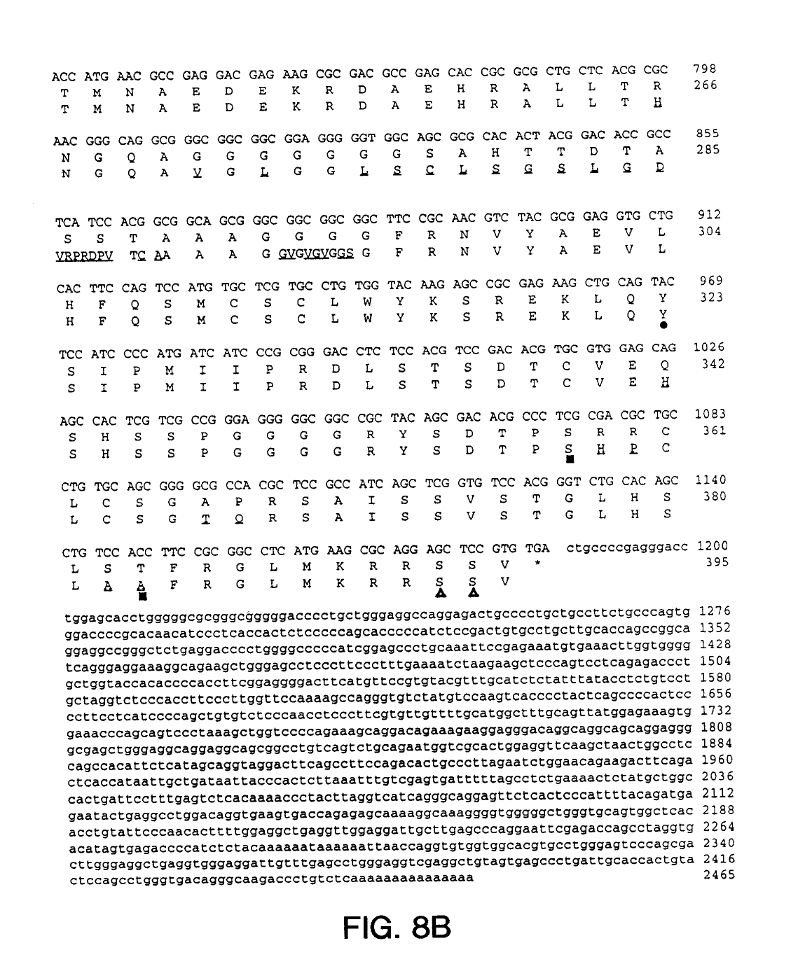

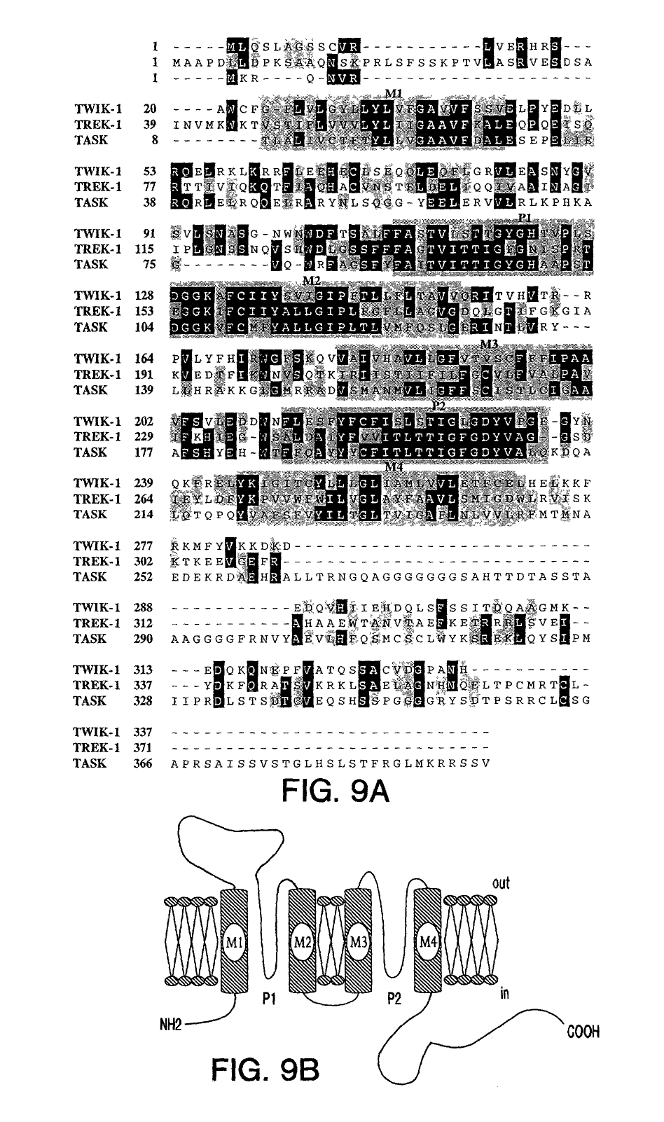

TWIK-1 and TREK-1 sequences were used to search related sequences in GenBank database by using the Blast alignment program. There were identified two mouse Expressed Sequence Tag (EST, accession numbers W36852 and W36914) that overlap and give a contig fragment of 560 bp whose deduced amino acid sequence presents significant similarity with TWIK-1 and TREK-1. corresponding DNA fragment was amplified by RT-PCR and used to screen a mouse brain CDNA library. Eight independent clones were isolated. The 1.8 kb cDNA insert of the longer one bears in its 5′ part an open reading frame (ORF) coding for a 405 amino acids polypeptide (FIG. 8). This ORF does not begin with an initiating methionine codon suggesting that the brain cDNA clones were partial. Ten additional positive clones were isolated from a mouse heart CDNA library. Analysis of their 5′ sequence showed that all these clones were not longer than the clones previously isolated from brain. The 5′ sequence has a very high GC content and is probably associated to secondary structures that could have promoted prematurate stops of RNA reverse transcription during the construction of both mouse cDNA libraries. To overcome this problem, the complete cDNA was cloned in another species. The DNA probe was used to screen a cDNA library from human kidney; a tissue that express both TWIK-1 and TREK-1 channels. Two hybridizing clones were characterized. Both contain an ORF of 1185 nucleotides encoding a 394 amino acids polypeptide (FIG. 8). The human protein sequence contains consensus sites for N-linked glycosylation (residue 53), and phosphorylation by protein kinase C (residues 358 and 383), tyrosine kinase (residue 323) and protein kinase A (residues 392 and 393). All these phosphorylation sites are located in the C-terminus part of the protein. Except for a 19 residues cluster (aa 276 to 294 in the human sequence), mouse and human proteins share a high overall sequence conservation (85% of identity) indicating that they probably are products of ortholog genes (FIG. 8). Sequence alignments presented in FIG. 9 clearly show that the cloned protein is a new member of the TWIK related K⁺ channel family. Like TWIK- 1 and TREK-1, TASK has four putative transmembrane segments (M1 to M4) and two P domains (P1 and P2) (FIGS. 9A and 9B). TASK is 58 amino acids longer than TWIK-1 and 24 amino acids longer than TREK-1 because its C-terminus is more extended.

Distribution of TASK

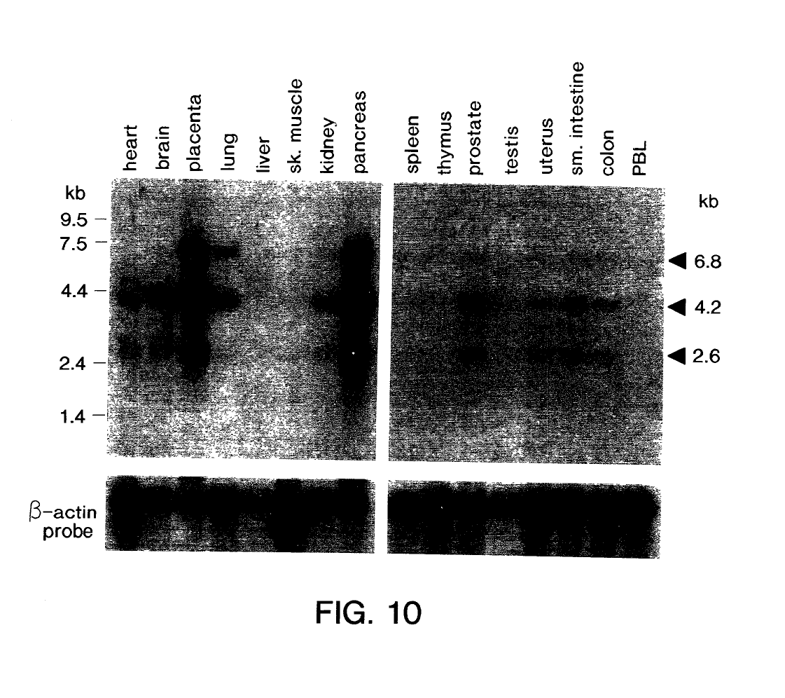

The expression of TASK in adult human and mouse tissues was examined by Northern blot analysis. Three different transcripts were detected in the human tissues with estimated sizes of 6.8, 4.2 and 2.6 kb (FIG. 10), the shorter one having the same size that the cloned cDNAs. The two other transcripts (4.2 and 6.8 kb) may result from alternate polyadenylation signals in the 3′ non-coding sequence and/or correspond to alternatively spliced or immature forms of the transcript. TASK is expressed in many different tissues but is particularly expressed in pancreas and placenta. Lower levels of expression were found in the brain>lung, prostate>heart, kidney>uterus, small intestine and colon.

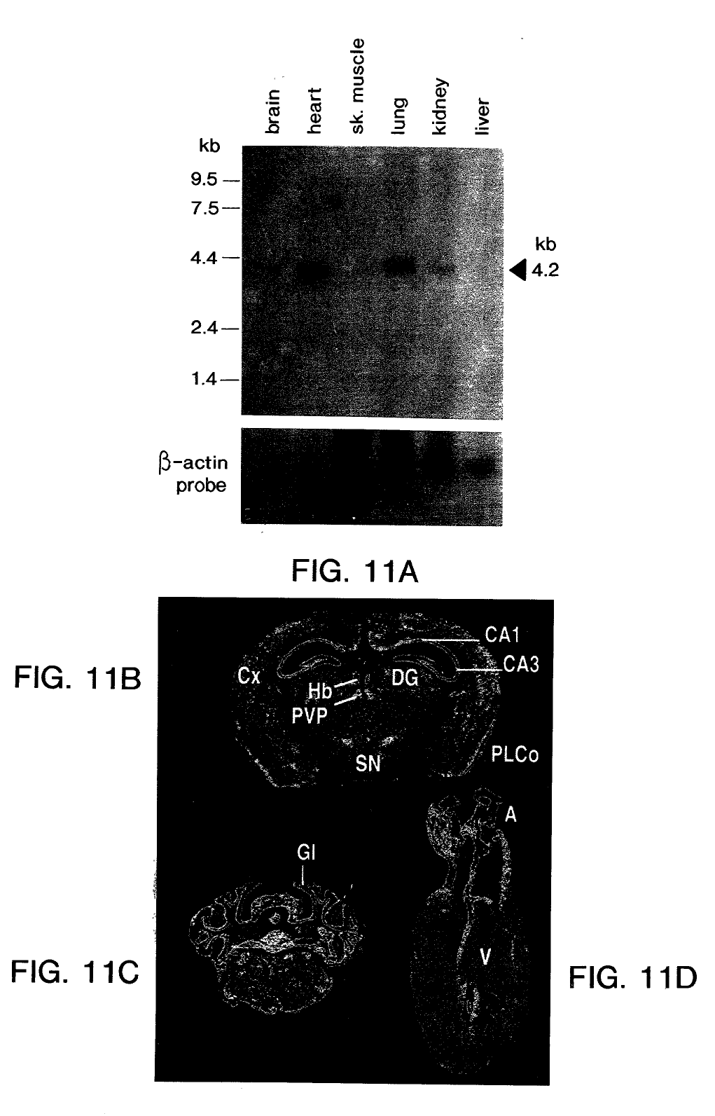

As shown in FIG. 11A, the TASK probe detected a single transcript in the mouse with an estimated size of 4.2 kb. TASK is expressed in the heart>lung>brain and kidney. No expression was seen in liver and skeletal muscle. The TASK distribution was further studied in adult mouse brain and heart by in situ hybridization. A wide and heterogeneous pattern of expression was obtained in the brain (FIGS. 11B and 11C). TASK MRNA was detected throughout the cell layers of the cerebral cortex, in the CA1-CA4 pyramidal cell layer, in the granule cells of the dentate gyrus, in the habenula, in the paraventricular thalamic nuclei, in the amyloid nuclei, in the substantia nigra and in the Purkinje and granular cells of the cerebellum. In the heart, a high level of TASK expression was found in the atria (FIG. 11D) while ventricular cells did not express this channel.

The TASK distribution was further studied in adult mouse brain and heart by in situ hybridization. In situ hybridization experiments were performed on adult Balb/c mice by using standard procedures (Fink et al., 1996b). An antisense oligonucleotide (48 mer, 5′-CACCAGCAGGTAGGTGAAGGTGCACACGATGAGAGCCAACGTGCGCAC-3′) complementary to the mouse cDNA sequence of TASK (from nucleotides 7 to 54) was used to detect the expression of TASK transcripts in frozen fixed brain sections (10 μm). The probe was 3′-end-labelled with (α-³³P)dATP. Sections were digested with 5 μg/ml of proteinase K for 15 min at 37° C., acetyled for 10 min in 0.25% acetic anhydre in 0.1 M triethanolamine. Hybridization was carried out overnight at 37° C. in 2X SSC, 50% formamide, 10% dextran sulfate, 1X Denhardt's solution, 5% sarcosyl, 500 μg denatured salmon sperm DNA, 250 mg/ml yeast tRNA, 20 mM dithiothreitol, and 20 mM NaPO₄ with 0.2 ng/ml of radiolabelled probe (specific activity=8.10₈ dpm/μg). Slides were then washed in 1X SSC before dehydratation, drying, and apposition to hyperfilm-βmax (Amersham) for 6 days. The specificity of labelling was verified by in situ hybridization using cold displacement of radioactive probe with a 500-fold excess of unlabelled oligonucleotide.

Biophysical Properties Of Task Currents

For functional studies, TASK cRNAs were injected into Xenopus oocytes. This was accomplished by subcloning a 2480 bp SmaI/XhoI fragment from pBS-hTASK containing 14 bp of 5′ UTR, the coding sequence and the entire 3′ UTR into the pEXO vector (Lingueglia et al., 1993) to give pEXO-TASK. Capped-cRNAs were synthesised in vitro from the linearized plasmid by using the T7 RNA polymerase (Stratagene). Xenopus laevis were purchased from CRBM (Montpellier, France). Preparation and cRNA injection of oocytes has been described elsewhere (Guillemare et al., 1992). Oocytes were used for electrophysiological studies 2 to 4 days following injection (20 ng/oocyte). In a 0.3 ml perfusion chamber, a single oocyte was impaled with two standard microelectrodes (1-2.5 MΩ resistance) filled with 3 M KCl and maintained under voltage clamp by using a Dagan TEV 200 amplifier, in standard ND96 solution (96 mM NaCl, 2 mM KCl, 1.8 mM CaCl₂, 2 mM MgCl₂, 5 mM HEPES, pH 7.4 with NaOH). In some experiments, NaCl was substituted with TMA Cl (Tetra Methyl Ammonium Chloride). Stimulation of the preparation, data acquisition, and analysis were performed using pClamp software (Axon instruments, USA). Drugs were applied externally by addition to the superfusate (flow rate: 3 ml/min) or intracellularly injected by using a pressure microinjector (Inject+Matic, Switzerland). All experiments were performed at room temperature (21-22° C.).

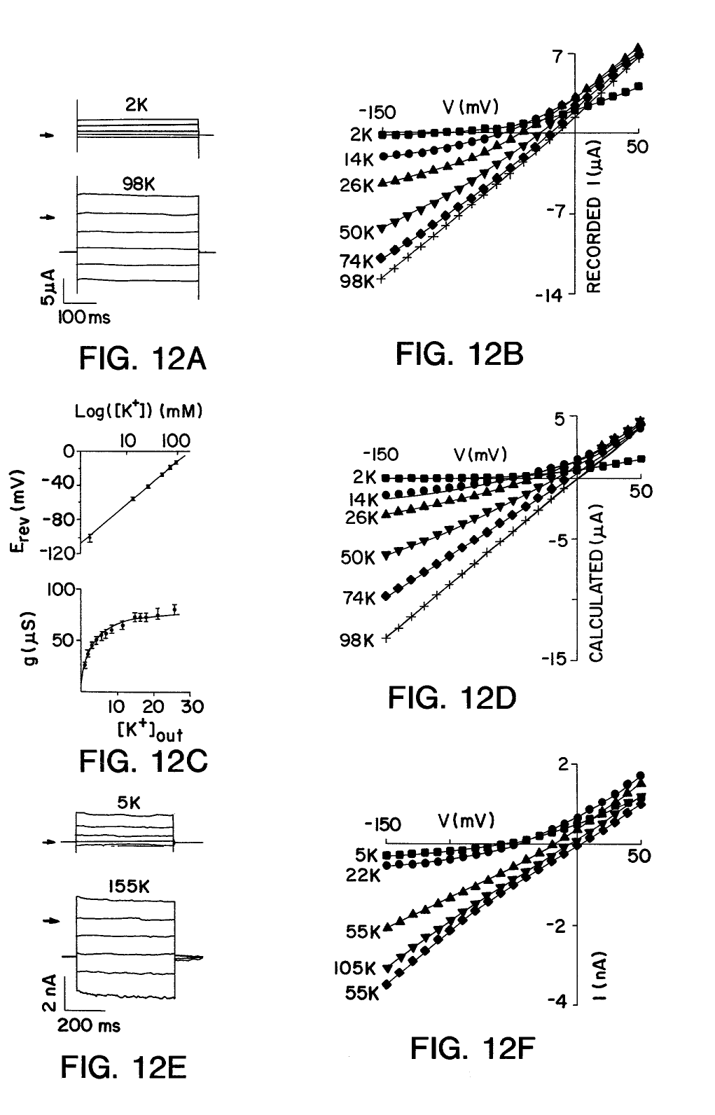

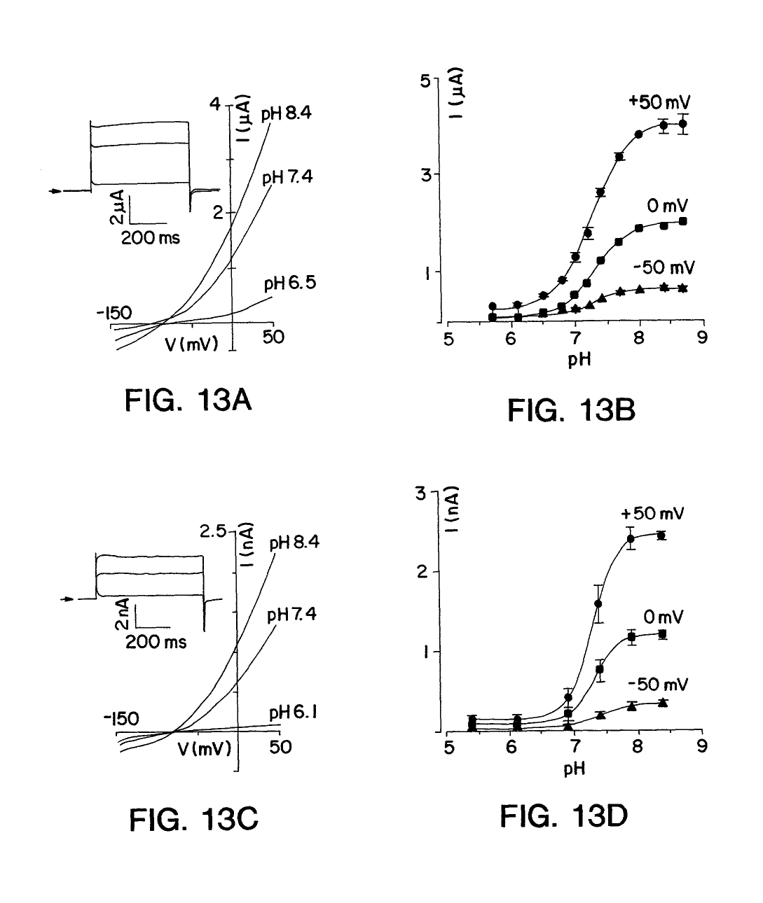

A non-inactivating current, not present in uninjected oocytes (not shown), was measured by two-electrode voltage-clamp (FIG. 12A). Activation kinetics of the TASK current are almost 10 instantaneous (under 10 ms). The current-voltage (I-V) relationship is outwardly-rectifying and almost no inward currents were recorded in the ND96 external medium containing 2 mM K⁺ (FIG. 12B). However, inward currents were revealed when the external K⁺ concentration ([K^{+]}ₒᵤₜ) was gradually increased to 98 mM K⁺ (FIGS. 12A and 12B). FIG. 12A presents the I-V relationships of the current in K⁺-rich solutions ranging from 2 mM to 98 mM. The relationship between the reversal potential and [K⁺]ₒᵤₜ was close to the predicted Nernst value (52.1 mV/decade, n=4) as expected for highly selective K⁺ channel (FIG. 12C, upper panel). On the other hand, external K⁺ enhanced the outward currents in a concentration-dependent manner as illustrated in FIG. 12C (lower panel). The half maximum activation by K⁺ was observed at a K_{0.5} of 2.06 mM. The theoretical I-V relationships in various [K⁺]ₒᵤₜ calculated according to the Goldman-Hodgkin-Katz current equation are shown in FIG. 12D. These I-V relationships are very close to the I-V relationships corresponding to recorded TASK currents (FIG. 12A). This strongly suggests that TASK currents show no rectification other than that predicted from the constant-field assumptions and that TASK lacks intrinsic voltage-sensitivity. The slight deviation between experimental and theoretical points are probably due to small endogenous chloride conductance and/or a K⁺ loading of the oocytes. It has been previously shown that oocytes expressing TWIK-1 or TREK-1 are more polarized that control oocytes, the resting membrane potential (Eₘ) reaching a value close to the K⁺ equilibrium potential (E_{K}). In oocytes expressing TASK, Eₘ was −85±0.8 mV (n=23, in standard ND96) instead of −44 ±2.6 mV (n=9) in non-injected oocytes. This result demonstrates that TASK, like other TWIK or TREK channels, is able to drive Eₘ close to E_{K}. The effect of various pharmacological agents on currents elicited by voltage pulses to +50 mV has been studied in TASK-expressing oocytes. Less than 20% of TASK currents were inhibited in the presence of quinine (100 μM), quinacrine (100 μM) or quinidine (100 μM). The “classical” K⁺ channels blockers tetraethylammonium (TEA, 1 mM) and 4-aminopyridine (4AP, 1 mnM) were also inactive. Cs⁺ (100 μM) induced a voltage-dependent block of 31±2% (n=4) of the inward current, recorded at −150 mV, in 50 mM external K⁺. In the same conditions, Ba²⁺ (100 μM) was ineffective with a variation of 6±1% (n=4) of the inward current.

Biophysical properties of TASK channel in transfected COS cells.

The 2480 bp SmaI/XhoI fragment of pBS-TASK was subcloned into the pCi plasmid (Promega) under the control of the cytomegaloviris promoter to give pCi-TASK. COS cells were seeded at a density of 70,000 cells per 35 mm dishes 24 h prior transfection. Cells were then transfected by the classical calcium phosphate precipitation method with 2 μg of pCI-TASK and 1 μg of CD8 plasmids. Transfected cells were visualized 48 h after transfection using the anti-CD8 antiboby coated beads method (Jurman et al., 1994). For electrophysiological recordings, the internal solution contained 150 mM KCl, 3 mm MgCl₂, 5 mM EGTA, and 10 mM HEPES at pH 7.2 with KOH, and the external solution 150 mM NaCl, 5 mM KCl, 3 mM MgCl₂, 10 mM HEPES at pH 7.4 with NaOH.

Untransfected cells did not express this K⁺ channel activity (not shown). FIG. 12E shows whole cell currents recorded in the mammalian COS cells transiently transfected with TASK, in external solutions containing 5 mM and 155 mM K⁺. The current were instantaneous and non-inactivating as in Xenopus oocytes. FIG. 12F presents the I-V relationships of TASK current in various external K⁺ concentrations. The currents show the same Goldman-Hodgkin-Katz type outward rectification as in oocytes.

Regulation of the TASK channel.

TASK currents were insensitive to internal Ca²⁺ changes obtained by injection of inositol triphosphate (IP3, 1 mM) or EGTA (100 mM), to the activation of adenyl cyclase by perfusion of IBMX (1 mM) and forskolin (10 μM), or to the activation of protein kinase C (PKC) by application of the phorbol ester PMA (40 nM). TASK currents were insensitive to the internal acidification or alkalisation obtained by injection of solutions at pH 2 or 8.7 respectively (n=3). However, their very interesting property is that they are highly sensitive to external pH. The current-potential relationships recorded from a TASK-expressing oocyte at pH 6.5, 7.4 and 8.4 are presented in FIG. 13A. For an external pH of 6.5, a drastic block was observed at all potentials while an activation was recorded at pH 8.4, also at all potentials. The inhibition and activation produced no modification of current kinetics (FIG. 13A, inset). The pH-dependence of the TASK channel is shown in FIG. 13B. For currents recorded at +50 mV, the inhibition by acidic pHs was characterised by an apparent pK of 7.34±0.04 units (n=3) and a Hill coefficient of 1.54±0.08 (n=3). For currents recorded at 0 and −50 mV, the pKs were 7.32±0.02 and 7.30±0.01 respectively (n=3) showing that the blocking effect of external protons is not voltage-dependent. The resting membrane potential of TASK-expressing oocytes was −84±1 mV (n=6) at pH 7.4 and shifted to −47±6 mV (n=6) at pH 6.4 (not shown). Finally, FIGS. 13C and 13D show that the strong pH sensitivity of TASK currents was also observed in transfected COS cells. A large inhibition or activation of the current was recorded, at all potentials, when pH was changed from 7.4 to 6.1 or 7.9 respectively (FIG. 13C). The kinetics of the current were unmodified at both pH (FIG. 6C, inset). FIG. 13D shows that the pH effects were also non voltage-dependent in COS cells. The external pH-dependence of TASK, at +50 mV, indicates a pK value of 7.29±0.03 (n=5) and a Hill coefficient of 1.57±0.07 (n=5). Currents recorded at 0 and −50 mV presented pKs of 7.29±0.04 (n=5) and 7.32±0.05 (n=4) respectively. 10% of the maximum current was obtained at pH 6.68±0.08 (n=4) and 90% at pH 7.66±0.05 (n=4). These results confirm that TASK is extremely sensitive to extracellular pH in the physiological range.

Other homologs of TWIK-1

Comparison of the complete sequence of TWIK-1 with the sequences of the Genbank data base allowed identification of at least five genes of Caenorhabditis elegans, which had been characterized in the context of the Nematode Sequencing project, which may encode additional structural homologues of TWIK-1. The alignment of two of these homologues with TWIK-1 is shown in FIG. 2b. The degree of similarity between the deduced protein sequences of C. elegans and TWIK-1 are approximately 55 to 60%. Amino acid sequence identities among the deduced polypeptide sequences range from 25 to 28%. Interestingly, the degree of similarity and amino acid sequence identity of the various C. elegans are not greater than what was determined for TWIK- 1. These results indicate that other TWIK-1 relates potassium channels may be present in the C. elegans genome and suggest that additional members of the TWIK-1 family of potassium channels may exist in mammals.

Unique structural features of TWIK-1 and TASK Family of Potassium Transport Channels.

This invention describes the isolation and the characterization a novel human K⁺ channel. This channel has an overall structure similarity with TWIK-1 and TREK-1 channels that suggests a common ancestral origin. Despite this similar structural organization, the amino acid identity between TASK and the two other mammalian related channels is very low (25-28%). Sequence homologies are no higher between TASK and a recently cloned Drosophila channel that also belongs to the structural TWIK channel class (Goldstein et al., 1996). The highest degree of sequence conservation is in the two P domains and the M2 segment. In these regions the amino acid identity reaches ˜50%. Like other TWIK-related channels, TASK contains an extended M1P1 interdomain. This peculiar domain has been shown to be extracellular in the case of TWIK- 1 and to be important for the self-association of two TWIK-1 subunits. The TWIK-1 homodimers are covalent because of the presence of an interchain disulfide bridge between cysteines 69 located in the M1P1 interdomain (Lesage et al., 1996b). This particular cysteine residue is conserved in TREK-1 (residue 93) but not in TASK strongly suggesting that TASK probably does not form covalent dimers as observed for TWIK1 (Lesage et al., 1996b) and TREK-1 (unpublished data).

The biophysical and regulation properties of TASK are unique. TWIK-1 has a mild inward rectification that involves an internal block by Mg²⁺ (Lesage et al, 1996a). TREK-1 expresses an outward rectification which seems to result from a voltage-sensitivity intrinsic to the channel protein (Fink et al., 1996b). In the case of TASK, the outward rectification observed at physiological external K⁺ concentrations can be approximated to the rectification predicted by the Goldman-Hodgkin-Katz current equation suggesting that this rectification simply results from the asymmetric concentrations of K⁺ on both sides of the membrane. In other words, this would mean that TASK lacks intrinsic voltage-sensitivity and behaves like a K⁺-selective “hole”. This behavior is unique among cloned mammalian K⁺ channels to the inventors' knowledge. Voltage- and time-independences are classical criteria to describe the so-called leak or background K⁺ channels. Some of these channels have been described in invertebrates, the best characterized of which being the S channels in Aplysia sensory neurones (Siegelbaum et al., 1982) and in vertebrates, for example in bullfrog sympathetic ganglia (Koyano et al., 1992), guinea-pig submucosal neurons (Shen et al., 1992), rat carotid bodies (Buckler, 1997), and guinea-pig ventricular myocytes (Backx and Marban, 1993). These channels are open at all membrane potentials and probably play a pivotal role in the control of the resting membrane potential and in the modulation of electrical activity of both neurones and cardiac cells. However, their lack of kinetics, voltage- and time-sensitivities, and their absence of a specific pharmacology has delayed their extensive electrophysiological and physiological characterization. Cloning of TASK, the first “true” background mammalian K⁺ channel should help to better characterize this peculiar functional family of K⁺ channels at the molecular level and identify specific and high affinity pharmacological agents that would block these channels and would facilitate analysis of their physiological roles.

TASK behaves as a K⁺-selective “hole” but this does not mean that its activity cannot be modulated. Unlike TWIK-1 and TREK-1 channels, its activity is not changed by activation of protein kinase A or C (Fink et al., 1996b; Lesage et al., 1996a). The probably very important property of TASK is that it is extremely sensitive to extracellular pH in the physiological range i e. between 6.5 and 7.8. The Hill coefficient of ˜1.6 found for the H ⁺ concentration dependence of the TASK current is consistent with the idea that the channel is formed by the assembly of 2 subunits as previously demonstrated for TWIK1. These 2 subunits would be in strong cooperative interactions in regards to H⁺.

The modulation by external protons probably has important implication for the physiological function of the TASK channel. Stimulus elicited pH shifts have been characterized in a wide variety of neural tissues by using extracellular pH-sensitive electrodes (reviewed in (Chesler, 1990; Chesler and Kaila, 1992)). They can be observed in physiopathological situations such as epileptiform activity and spreading depression in which acid shifts are usually preceded by alkaline transients (Siesjö et al., 1985; Nedergaard et al., 1991). They can be observed of course in ischemia where large acidifications of the extracellular medium have been recorded (Kraig et al., 1983; Mutch and Hansen, 1984). However, they can also be observed in physiological conditions such as electrical stimulation of Schaeffer collateral fibers in the hippocampal slice (Krishtal et al., 1987), or light stimulation of the retina (Borgula et al., 1989; Yamamoto et al., 1992), or parallel fibers in cerebellum (Kraig et al., 1983). All these pH-shifts correspond to bursts of H⁺ or OH⁻ creating small pH variations from the external physiological pH value of 7.4 in the alkaline or acidic direction (up to 0.3 pH units) and are rapid, in the second to 30 seconds range. They might actually be larger in range or shorter in time course in the vicinity of the synaptic cleft. A particularly interesting issue is whether these relatively small activity-dependent pH changes have significant modulatory effects. In other words, does H⁺ serve a transmitter role in the nervous system? The discovery of this new TASK channel that can fully open or close within a range of only 0.5 pH unit around the physiological pH (7.4) will certainly strengthen the idea that pH could be a natural modulator of neuronal activity (Chesler and Kaila, 1992).

From the above description it will seem that the present invention relates to an isolated, purified nucleic acid molecule that codes for a protein constituting a potassium channel of the TWIK-1 family or exhibiting the properties and structure of the type of the TWIK-1 channel described above.

More specifically, the said nucleic acid molecule codes for the TWIK-1 protein, the amino acid sequence of which is represented in the attached sequence list as number SEQ ID NO: 2, or TASK, represented in the attached sequence list as number SEQ ID NO: 4, or functionally equivalent derivatives of these sequences that possess the distinguishing structural features of the TWIK-1 family of potassium transport proteins. Such derivatives can be obtained by modifying and or suppressing one or more amino acid residues of this sequence, as long as this modification and/or suppression does not modify the functional properties of the TWIK-1 potassium channel of the resultant protein. The sequence of a DNA molecule coding for this protein is more specifically the molecule coding for TWIK-1, represented in the attached sequence list as number SEQ ID NO: 1 or TASK, represented in the attached sequence list as number SEQ ID NO: 3.

The invention also relates to a vector containing a molecule of the aforementioned nucleic acid sequences, as well as a procedure for production or expression in a cellular host of a protein constituting a TWIK- 1 potassium channel or a channel of the same family as TWIK- 1.

A procedure for production of a protein constituting a TASK potassium channel or exhibiting the properties and structure of the type of the TASK channel composes transferring a nucleic acid molecule of the invention or a vector containing the said molecule into a cellular host, culturing the transformed cellular host obtained in the preceding step under conditions enabling the production of potassium channels exhibiting the properties of TASK, and isolating the proteins constituting the potassium channels of the TASK family.

A procedure for the expression of a TASK potassium channel or a potassium channel of the same family as TASK comprises transferring a nucleic acid molecule of the invention or a vector containing the said molecule into a cellular host, and culturing the cellular host obtained under conditions enabling the expression of potassium channels of the TASK family. The cellular host employed can be selected from among the prokaryotes or the eukaryotes, and notably from among the bacteria, the yeasts, mammal cells, plant cells or insect cells. The vector used is selected in relation to the host into which it will be transferred; it can be any vector such as a plasmid. The invention also relates to the transferred cells expressing the potassium channels exhibiting the properties and structure of the type of the TASK channel obtained in accordance with the preceding procedures.

The cells expressing TASK potassium channels or channels exhibiting the properties and structure of the type of the TASK channels obtained in accordance with the preceding procedures are useful for the screening of substances capable of modulating the activity of the individual members of the TASK family of potassium channels. This screening is carried out by bringing into contact variable amounts of a substance to be tested with cells expressing the TASK channel or potassium channels exhibiting the properties and structure of the type of the TASK channels, then determining the effects of said substance on the currents of the potassium channels of these channels.

This screening procedure makes it possible to identify drugs that may be useful in the treatment of diseases of the heart or of the nervous system. Diseases involving the potassium channels and thus likely to involve the channels of the TASK family are, for example, epilepsy, heart (arrhythmias) and vascular diseases, neurodegenerative diseases, especially those associated with ischemia or anoxia, the endocrine diseases associated with anomalies of hormone secretion, muscle diseases.

An isolated, purified nucleic acid molecule coding for a protein of the TASK family of potassium channel or a vector including this nucleic acid molecule or a cell expressing the potassium channel polypeptide, are also useful for the preparation of transgenetic animals. These can be animals supra-expressing said channels, but especially so-called knock-out animals, i.e., animals presenting a deficiency of these channels; these transgenetic animals are prepared by methods known to the experts in the field, and enable the preparation of live models for studying animal diseases associated with the TASK family of channels.

The nucleic acid molecules of the invention or the cells transformed by said molecule can be used in genetic therapy strategies to compensate for a deficiency in the potassium channels at the level of one or more tissues of a patient. The invention thus also relates to a medication containing nucleic acid molecules of the invention or cells transformed by said molecule for the treatment of disease involving the potassium channels.

The present invention also has as its object a new family of K+ channels, of which TWIK-1 and TASK are members, which polypeptides are present in most human tissues, but especially abundant in the brain and the heart, and which exhibit the properties and structure of the type of those of the TWIK-1 channels described above. Thus, the invention relates to an isolated, purified protein whose amino acid sequence is represented in the attached sequence list as number SEQ ID NO: 4 or SEQ ID NO: X, or a functionally equivalent derivative of these sequences. Such derivatives can be obtained by modifying and/or suppressing one or more amino acid residues of this sequence or by segmenting this sequence, as long as this modification and/or suppression or deletion of a fragment does not modify the functional properties of the TASK type potassium channel of the resultant protein.

Proteins comprising a TASK type potassium channel are useful for the manufacture of medications intended for the treatment or prevention of diseases involving dysfunction of the potassium channels.

Polyclonal or monoclonal antibodies directed against a protein constituting a TASK type potassium channel can be prepared by conventional methods described in the literature. These - antibodies are useful for investigating the presence of potassium channels of the TASK family in different human or animal tissues, but can also be applied for the in vivo inhibition or activation of TASK type potassium channels.

Materials and Methods

Cloning of TASK and RNA analysis

TWIK-1 and TREK-1 were used to search homologues in gene databases by using the tblastn sequence alignment program (Altschul et al., 1990). Translation of two overlapping EST sequences (GenBank accession numbers W36852 and W36914) in one frame presented significant sequence similarities with TWIK- 1 and TREK-1. A 560 bp DNA fragment was amplified by PCR from mouse brain poly(A)⁺ cDNAs and subcloned into pBluescript (Stratagene) to give pBS-852/914. This fragment was ³²P-labelled and used to screen mouse brain and heart cDNA libraries. Filters were hybridized and washed as previously described (Fink et al., 1996b). Eight positive clones from brain and ten from heart were obtained. CDNA inserts were characterized by restriction analysis and by partial or complete sequencing on both strands by the dideoxy nucleotide chain termination method using an automatic sequencer (Applied Biosystems). All the clones were shown to only contain a partial ORF. The cDNA insert of the longer mouse clone (designated pBS-mTASK) was ³²P-labelled and used to screen a human kidney cDNA library. Two independent hybridizing clones were isolated and sequenced. Both clones (2.5 kb long) were shown to contain the full-length ORF. The longer one was designated pBS-hTASK.

For Northern blot analysis, poly(A)⁺ RNAs were isolated from adult mouse tissues and blotted onto nylon membranes as previously described (Lesage et al., 1992). The blot was probed with the ³²P-labelled insert of pBS-mTASK in 50% Formamide, 5X SSPE (0.9 M sodium chloride, 50 mM sodium phosphate (pH 7.4), 5 mM EDTA), 0.1% SDS, 5X Denhardt's solution, 20 mM potassium phosphate (pH 6.5) and 250 μg denatured salmon sperm DNA at 50° C. for 18 h and washed stepwise at 55° C. to a final stringency of 0.2X SSC, 0.3% SDS. For hybridization of human multiple tissues Northern blots from Clontech, the procedure was identical except that the probe was derived from pBS-hTASK. The cDNA insert of pBS-hTASK contains different repeat sequences (AluJb, MIR and (CGG)n) in the untranslated regions (UTR) and a SmaI/ApaI restriction fragment of 1390 bp spanning the coding sequence was chosen as a probe that does not contain these repeats.

### LIST OF SEQUENCES

INFORMATION REGARDING SEQ ID NO: 1

I - CHARACTERISTIC OF THE SEQUENCE:

A) LENGTH:

B) TYPE

C) STRAND NUMBER

D) CONFIGURATION

II - TYPE OF MOLECULE:

XI - SEQUENCE DESCRIPTION: SEQ ID NO: 1

INFORMATION REGARDING SEQ ID NO: 2

I - CHARACTERISTIC OF THE SEQUENCE:

A) LENGTH:

B) TYPE

C) STRAND NUMBER

D) CONFIGURATION

II - TYPE OF MOLECULE:

XI - SEQUENCE DESCRIPTION: SEQ ID NO: 2

INFORMATION REGARDING SEQ ID NO: 3

I - CHARACTERISTIC OF THE SEQUENCE:

A) LENGTH:

B) TYPE

C) STRAND NUMBER

D) CONFIGURATION

II - TYPE OF MOLECULE:

XI - SEQUENCE DESCRIPTION: SEQ ID NO: 3

INFORMATION REGARDING SEQ ID NO: 4

I - CHARACTERISTIC OF THE SEQUENCE:

A) LENGTH:

B) TYPE

C) STRAND NUMBER

D) CONFIGURATION

II - TYPE OF MOLECULE:

XI - SEQUENCE DESCRIPTION: SEQ ID NO: 4

All references cited in this text are expressly incorporated herein by reference.

### REFERENCES

Altschul, S. F., Gich, W., Miller, W., Myers, E. W. and Lipman, D. J. (1990) Basic local alignment search tool. J. Mol. Biol., 215, 403-410.

Backx, P. H. and Marban, E. (1993) Background potassium current active during the plateau of the action potential in Guinea-pig ventricular myocytes. Circulation Res., 72, 890-900.

Barhanin, J., Lesage, F., Guillemare, E., Fink, M., Lazdunski, M. and Romey, G. (1996) KvLQT1 and IsK (minK) proteins associate to form the IKs cardiac potassium current. Nature., 384, 78-80.

Borgula, G. A., Karwoski, C. J. and Steinberg, R. H. (1989) Light-evoked changes in extracellular pH in frog retina. Vision Res., 29, 1069-1077.

Buckler, K. J. (1997) A novel oxygen-sensitive potassium current in rat carotid body type I cells. J. Physiol. (London)., 498, 649-662.

Chesler, M. (1990) The regulation and modulation of pH in the nervous system. Prog. Neurobiol., 34, 401-427.

Chesler, M. and Kaila, K. (1 992) Modulation of pH by neuronal activity. Trends Neurosci., 15, 386-402.

Doupnik, C. A., Davidson, N. and Lester, H. A. (1995) The inward rectifier potassium channel family. Curr. Opin. Neurobiol., 5, 268-277.

Fakler, B. and Ruppersberg, J. P. (1996) Functional and molecular diversity classifies the family of inward-rectifier K⁺ channels. Cell. Physiol. Biochem., 6, 195-209.

Fink, M., Duprat, F., Lesage, F., Heurteaux, C., Romey, G., Barhanin, J. and Lazdunski, M. (1996a) A new K⁺ channel β subunit to specifically enhance Kv2.2 (CDRK) expression. J. Biol. Chem., 271, 26341-26348.

Fink, M., Duprat, F., Lesage, F., Reyes, R., Romey, G., Heurteaux, C. and Lazdunski, M. (1996b) Cloning, functional expression and brain localization of a novel unconventional outward rectifier K⁺ channel. EMBO J., 15, 6854-6862.

Goldstein, S. A. N., Price, L. A., Rosenthal, D. N. and Pausch, M. H. (1996) ORK1, a potassium-selective leak channel with two pore domains cloned from Drosophila melanogaster by expression in Saccharomyces cerevisiae. Proc. Natl. Acad. Sci. USA., 93, 13256-13261.

Guillemare, E., Honore, E., Pradier, L., Lesage, F., Schweitz, H., Attali, B., Barhanin, J. and Lazdunski, M. (1992) Effects of the level of messenger RNA expression on biophysical properties, sensitivity to neurotoxins, and regulation of the brain delayed-rectifier K⁺ channel Kv1.2. Biochemistry., 31, 12463-12468.

Heginbotham, L., Lu, Z., Abramson, T. and Mackinnon, R. (1994) Mutations in the K⁺ channel signature sequence. Biophys. J., 66, 1061-1067.

Hille, B. (1992) in (ed.) Ionic channels of excitable membranes. Sinauer Associates Inc., Sunderland, Mass. pp. 1-607.

Inagaki, N., Gonoi, T., Clement, J. P., Wang, C. Z., Aguilarbryan, L., Bryan, J. and Seino, S. (1996) A family of sulfonylurea receptors determines the pharmacological properties of ATP-sensitive K⁺ channels. Neuron., 16, 1011-1017.

Jan, L. Y. and Jan, Y. N. (1994) Potassium channels and their evolving gates. Nature., 371, 119-122.

Jurman, M. E., Boland, L. M. and Yellen, G. (1994) Visual identification of individual transfected cells for electrophysiology using antibody-coated beads. BioTechniques., 17, 876-881.

Knaus, H. G., Folander, K., Garciacalvo, M., Garcia, M. L., Kaczorowski, G. J., Smith, M. and Swanson, R. (1994) Primary sequence and immunological characterization of beta-subunit of high conductance Ca^{2 +}-activated K⁺ channel from smooth muscle. J. Biol. Chem., 269, 17274-17278.

Kohler, M., Hirschberg, B., Bond, C. T., Kinzie, J. M., Marrion, N. V., Maylie, J. and Adelman, J. P. (1996) Small-conductance, calcium-activated potassium channels from mammalian brain. Science., 273, 1709-1714.

Koyano, K., Tanaka, K. and Kuba, K. (1992) A patch-clamp study on the muscarine-sensitive potassium channel in bullfrog sympathetic ganglion cells. J. Physiol. (London)., 454, 231-246.

Kraig, R. P., Ferreira-Filho, C. R. and Nicholson, C. (1983) Alkaline and acid transients in cerebellar microenvironment. J. Neurophysiol., 49, 831-851.

Krishtal, O. A., Osipchuk, Y. V., Shelest, T. N. and Smimoff, S. V. (1987) Rapid extracellular Ph transients related to synaptic transmission in rat hippocampal slices. Brain Res., 436, 352-356.

Kyte, J. and Doolittle, R. (1982) A simple model for displaying the hydropathic character of a protein. J. Mol. Biol., 157, 105-106.

Lesage, F., Attali, B., Lazdunski, M. and Barhanin, J. (1992) Developmental expression of voltage-sensitive K⁺ channels in mouse skeletal muscle and C2C12 cells. FEBS Lett., 310, 162-166.

Lesage, F., Guillemare, E., Fink, M., Duprat, F., Heurteaux, C., Fosset, M., Romey, G., Barhanin, J. and Lazdunski, M. (1995) Molecular properties of neuronal G-protein-activated inwardly rectifying K³⁰ channels. J. Biol. Chem., 270, 28660-28667.

Lesage, F., Guillemare, E., Fink, M., Duprat, F., Lazdunski, M., Romey, G. and Barhanin, J. (1996a) TWIK-1, a ubiquitous human weakly inward rectifying K⁺ channel with a novel structure. EMBO J., 15, 1004-1011.

Lesage, F., Lauritzen, I., Duprat, F., Reyes, R., Fink, M., Heurteaux, C. and Lazdunski, M. (1997) The structure, function and distribution of the mouse TWIK-1 K⁺ channels. FEBS Lett., 402, 28-32.

Lesage, F., Reyes, R., Fink, M., Duprat, F., Guillemare, E. and Lazdunski, M. (1996b) Dimerization of TWIK-1 K⁺ channel subunits via a disulfide bridge. EMBO J., 15, 6400-6407.

Lingueglia, E., Voilley, N., Waldmann, R., Lazdunski, M. and Barbry, P. (1993) Expression cloning of an epithelial amiloride-sensitive Na⁺ channel. A new channel type with homologies to Caenorhabditis elegans degenerins. FEBS Lett., 318, 95-99.

MacCobb, D. P., Fowler, N. L., Featherstone, T., Lingle, C. J., Saito, M., Krause, J. E. and Salkoff, L. (1995) A human calcium-activated potassium channel gene expressed in vascular smooth muscle. Am. J. Physiol.-Heart Circ. Physiol., 38, H767-H777.

MacManus, O. B., Helms, L. M. H., Pallanck, L., Ganetzki, B., Swanson, R. and Leonard, R. J. (1995) Neuron., 15, 645-650.

Mutch, W. A. C. and Hansen, A. J. (1984) Extracellular pH changes during depression and cerebral ischemia: mechanisms of brain pH regulation. J. Cereb. Blood Flow Metab., 4, 17-27.

Nedergaard, M., Kraig, R. P., Tanabe, J. and Pulsinelli, W. A. (1991) Dynamics of interstitial and intracellular pH in evolving brain infarct. Am. J. Physiol., 260, R581-R588.

Pongs, O. (1992) Molecular biology of voltage-dependent potassium channels. Physiol. Rev., 72, S69-S88.

Pongs, O. (1995) Regulation of the activity of voltage-gated potassium channels by beta subunits. Semin. Neurosci., 7, 137-146.

Rudy, B. (1988) Diversity and ubiquity of K⁺ channels. Neuroscience., 25, 729-749.

Sanguinetti, M. C., Curran, M. E., Zou, A., Shen, J., Spector, P. S., Atkinson, D. L. and Keating, M. T. (1996) Coassembly of KvLQT1 and MinK (IsK) proteins to form cardiac I_{KS} potassium channel. Nature., 384, 80-83.

Shen, K. Z., North, R. A. and Surprenant, A. (1992) Potassium channels opened by noradrenaline and other transmitters in excised membrane patches of guinea-pig submucosal neurones. J. Physiol. (London)., 445, 581-599.

Siegelbaum, S. A., Camardo, J. S. and Kandel, E. (1982) Serotonin and cyclic AMP close single K⁺ channels in Aplysia sensory neurones. Nature., 229, 413-417.

Siesjö, B. K., von Hanwehr, R., Nerglius, G., Nevander, G. and Ingvar, M. (1985) Extra- and intracellular pH in the brain during seizures and in the recovery period following the arrest of seizure activity. J. Cereb. Blood Flow Metab., 5, 47-57.

Takumi, T., Ohkubo, H. and Nakanishi, S. (1988) Cloning of a membrane protein that induces a slow voltage-gated potassium current. Science., 242, 1042-1045.

Yamamoto, F., Borgula, G. A. and Steinberg, R. H. (1992) Effects of light and darkness on pH outside rod photoreceptors in the cat retina. Exp. Eye Res., 54, 685-697.

While present invention has been described with reference to specific embodiments thereof, it will be appreciated that numerous variations, modifications, and embodiments are possible, and accordingly, all such variations, modifications and embodiments are to be regarded as being within the spirit and scope of the present invention.

1. An isolated and purified nucleic acid molecule coding for a protein having a potassium (K⁺) permeable membrane, comprising more than one P domains and three, four, five or more than six transmembrane segments.

2. The nucleic acid molecule of claim 1 coding for a protein wherein the number of P domains is two and the number of transmembrane segments is four.

3. The nucleic acid molecule of claim 1 which is human.

4. The nucleic acid molecule of claim 1 which is a cDNA copy of a 2.6 kilobase transcript expressed at high levels in the pancreas and placenta, and at lower levels in the brain, lung, prostate, heart, kidney, uterus small intestine and colon.

5. The nucleic acid sequence of claim 1 which codes for a protein which comprises the sequence represented by SEQ ID No. 4.

6. The isolated and purified nucleic acid sequence of claim 1 which codes for a protein which comprises the s equence represented by SEQ ID No. 4 or the functionally equivalent sequence thereof which comprises two P domains and four transmembrane segments.

7. An isolated and purified nucleic acid sequence of claim 2 which comprises our open reading frame (ORF) of 1185 nucleotides.

8. The isolated and purified nucleic acid sequence of claim 7 which is human.

9. An isolated and purified protein having a potassium (K⁺) permeable membrane comprising more than one P domain and three, four, five or more than six transmembrane segments.

10. The protein of claim 9 wherein the number of P domains is two and the number of transmembrane segments is four.

11. The protein of claim 10 in which the potassium transport channel exhibits outward rectification when the extracellular concentration of potassium is 2 mM and no rectification when the extracellular potassium is 98 mM, thereby evidencing lack of intrinsic voltage sensitivity

12. The protein of claim 10 in which the potassium transport channel lacks intrinsic voltage, lacks kinetics voltage- and time sensitivities, thereby evidencing characteristics of background conductance.

13. The protein of claim 9 in which the activity of the potassium transport channel is regulated by extracellular pH in a physiological range of 6.5 and 7.8.

14. The protein of claim 13 which the potassium channel exhibits 10% transport activity at pH 6.7, and 90% transport activity at pH 7.7.

15. The protein of claim 14 which is human.

16. The protein of claim 15 which comprises the sequence represented by SEQ ID No. 4.

17. A method of screening for substances capable of modulating the activity of the potassium transport channel encoded by the nucleic acid sequence of claim 1 comprising contacting pre-selected amounts of the substance to be tested with cells expressing the potassium transport channel, measuring the effects of said substance on the transport functions of the potassium transport channel, and identifying the substance that has a positive or negative effect on potassium channel activity.

18. A substance, identified by the method of claim 17 that is competent to positively or negatively influence the transport functions of a potassium transport channel.

19. A method for identifying genetic polymorphisms in the locus comprising the nucleic acid sequence of claim 1 by hybridizing DNA samples under stringent conditions with a probe comprising the isolated nucleic acid sequence encoding the potassium transport channel.

20. The method of claim 19 where the probe is hybridized to intact chromosomes in situ.

21. The method of claim 20 where the probe is hybridized with Southern blots of genomic DNA digested with a restriction endonuclease.

22. The method of claim 17 wherein the nucleic acid sequence encodes a protein in which the potassium transport channel lacks kinetics, voltage- and time-sensitivities, thereby evidencing characteristics of background conductance.

23. A substance identified by the method of claim 22 which is competent to positively or negatively influence the transport functions of a potassium transport channel.

24. A self replicating vector comprising the nucleic acid molecule of claim 1.

25. A cell transformed with the vector of claim 24, which cell is selected from the group consisting of prokaryotes and eukaryotes.

26. The transformed cell of claim 25 which is a yeast, insect cell, plant cell or mammalian cell.

27. The transformed cell of claim 25 which is a bacteria.

28. A method for the expression and isolation of a potassium transport channel encoded by the nucleic acid molecule of claim 1 in a competent host cell comprising transferring the vector of claim 24 into a competent host cell, culturing said host cell under conditions allowing the production of the potassium transport channel, and isolating and purifying the polypeptide comprising the potassium transport channel.

29. A transgenic animal which comprises the nucleic acid sequence of claim 1 encoding a potassium transport channel.

30. The transgenic animal of claim 29 in which the nucleic acid sequence encoding the potassium transport channel is non-human.

31. The transgenic animal of claim 29 which overexpresses the potassium transport channel encoded by the nucleic acid sequence represented by SEQ ID No. 3.

32. The transgenic animal of claim 29 which is deficient in the expression of the potassium transport channel encoded by the nucleic acid sequence represented by SEQ ID No. 3.

33. A pharmaceutical composition for the treatment of diseases caused by a defective potassium transport or a deficiency of the potassium transport protein comprising the nucleic acid of claim 1 or the transformed cells of claim 25 in one or more tissues having a defective potassium transport function under conditions which allow for the expression of the potassium transport channel in said tissue.
